(19) United States
(12) Patent Application Publication
(10) Pub. No.: US 2010/0168182 A1
(43) Pub. Date: Jul. 1, 2010

(54) COMPOUNDS AND COMPOSITIONS AS KINASE INHIBITORS

(75) Inventors: Yuan Mi, Shanghai (CN); Pamela Albaugh, Carlsbad, CA (US); Yi Fan, Poway, CA (US); Ha-Soon Choi, San Diego, CA (US); Zuosheng Liu, San Diego, CA (US); Shenlin Huang, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/518,831

(22) PCT Filed: Dec. 11, 2007
(86) PCT No.: PCT/US2007/025447

§ 371 Date: Mar. 2, 2010

Related U.S. Application Data
(60) Provisional application No. 60/869,548, filed on Dec. 11, 2006.

(51) Int. Cl. A61K 31/404; A61K 31/4439; A61K 31/427; A61K 31/422; A61K 31/4178; A61K 31/4155; C07D 401/14; C07D 417/14; C07D 413/14; C07D 403/14; C07D 403/06; A61P 35/00; C07D 405/14

(52) U.S. Cl. 514/339; 514/371; 514/380; 514/397; 514/407; 514/414; 546/277.7; 548/181; 548/245; 548/364.7; 548/468

(57) ABSTRACT

The invention provides a novel class of compounds, pharmaceutical compositions comprising such compounds and methods of using such compounds to treat or prevent diseases or disorders associated with abnormal or deregulated kinase activity, particularly TrkA, TrkB, TrkC, PDGFR and c-kit.

### CROSS-REFERENCE TO RELATED APPLICATIONS

This application claims the benefit of priority to U.S. Provisional Patent Application No. 60/869,548, filed 11 Dec. 2006. The full disclosure of this application is incorporated herein by reference in its entirety and for all purposes.

### BACKGROUND OF THE INVENTION

1. Field of the Invention

The invention provides a novel class of compounds, pharmaceutical compositions comprising such compounds and methods of using such compounds to treat or prevent diseases or disorders associated with abnormal or deregulated kinase activity, particularly TrkA, TrkB, TrkC, PDGFR and c-kit.

2. Background

The protein kinases represent a large family of proteins, which play a central role in the regulation of a wide variety of cellular processes and maintaining control over cellular function. A partial, non-limiting, list of these kinases include: receptor tyrosine kinases such as platelet-derived growth factor receptor kinase (PDGF-R), the nerve growth factor receptor, Trk-A, -B and -C, and the fibroblast growth factor receptor, FGFR3; non-receptor tyrosine kinases such Abl and the fusion kinase BCR-Abl, Lck, Csk, Fes, BTK, Bmx and c-src; and serine/threonine kinases such as Aurora, c-RAF, SGK, MAP kinases (e.g., MKK4, MKK6, etc.) and SAPK2α and SAPK2β. Aberrant kinase activity has been observed in many disease states including benign and malignant proliferative disorders as well as diseases resulting from inappropriate activation of the immune and nervous systems.

The novel compounds of this invention inhibit the activity of one or more protein kinases and are, therefore, expected to be useful in the treatment of kinase-associated diseases such as pancreatic cancer, papillary thyroid carcinoma and neuroblastoma.

### SUMMARY OF THE INVENTION

In one aspect, the present invention provides compounds of Formula I:

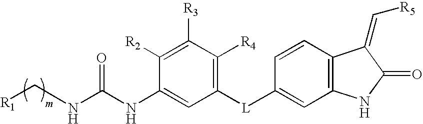

in which:

L is selected from O, NH and S;

m is selected from 0 and 1;

R₁ is selected from phenyl, pyridinyl, furanyl, isoxazolyl, pyrazolyl and thiazolyl; wherein said phenyl, pyridinyl and furanyl of R₁, can be optionally substituted with 1 to 3 radicals independently selected from halo, C₁₋₄alkyl, halo-substituted-C₁₋₄alkyl, C₁₋₄alkoxy, halo-substituted-C₁₋₄alkoxy, cyano-substituted-C₁₋₄alkyl, —XR₆ and —NR₇ₐR_{7b}; wherein X is selected from a bond and C₁₋₄alkylene; R₆ is selected from C₃₋₈heterocycloalkyl and C₃₋₁₂cycloalkyl; wherein R₆ is optionally substituted with 1 to 2 radicals independently selected from cyano and C₁₋₄alkyl; and R₇ₐ and R_{7b} are independently selected from hydrogen and C₁₋₄alkyl; wherein said isoxazolyl, pyrazolyl and thiazolyl of R₁, can be optionally substituted with 1 to 2 radicals independently selected from halo, C₁₋₄alkyl, halo-substituted-C₁₋₄alkyl, C₁₋₄alkoxy, halo-substituted-C₁₋₄alkoxy and cyano-substituted-C₁₋₄alkyl;

R₂ is selected from methyl, halo, methoxy and cyano;

R₃ is selected from methyl, halo, methoxy and cyano;

R₄ is selected from methyl, halo, methoxy and cyano;

R₅ is selected from pyrrolyl and imidazole; wherein said pyrrolyl or imidazolyl of R₅ can be optionally substituted with 1 to 2 radicals independently selected from C₁₋₄alkyl, cyano, —C(O)OR₈ₐ, —C(O)NR₈ₐR_{8b}, —X₂NR₈ₐX₂NR₈ₐR_{8b} and —C(O)NR₈ₐX₂NR₈ₐR_{8b}; wherein said alkyl substituents of R₅ are optionally substituted with —NR₉ₐR_{9b}; wherein R₈ₐ, R_{8b}, R₉ₐ and R_{9b} are each independently selected from hydrogen and C₁₋₄allyl; each X₂ is independently C₁₋₄alkylene; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which inhibition of kinase activity, particularly, TrkA, TrkB, TrkC, PDGFR and c-kit activity, can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which kinase activity, particularly TrkA, TrkB, TrkC, PDGFR and c-kit activity, contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group.

“Heteroaryl” is as defined for aryl above where one or more of the ring members is a heteroatom. For example heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, pyrrolidinyl-2-one, piperazinyl, piperidinyl, piperidinylone, 1,4-dioxa-8-aza-spiro[4.5]dec-8-yl, etc.

“Halogen” (or halo) preferably represents chloro or fluoro, but may also be bromo or iodo.

“Mutant forms of BCR-Abl” means single or multiple amino acid changes from the wild-type sequence. Over 22 mutations have been reported to date with the most common being G250E, E255V, T315I, F317L and M351T.

“NTKR1” is the gene name equivalent to TrkA protein; “NTKR2” is the gene name equivalent to TrkB protein; and “NTKR3” is the gene name equivalent to TrkC protein.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of kinase related disease, particularly TrkA, TrkB, TrkC, PDGFR and c-kit. For example, inhibitors of TrkA, TrkB and TrkC are useful for the treatment of pancreatic cancer, papillary thyroid carcinoma and neuroblastoma.

In one embodiment, with reference to compounds of Formula I, R₅ is selected from pyrrolyl and imidazolyl; wherein said pyrrolyl or imidazolyl of R₅ can be optionally substituted with 1 to 2 radicals independently selected from C₁₋₄alkyl, cyano, —C(O)OCH₃, —C(O)NH and —C(O)NH(CH₂)₂N(C₂H₅)₂; wherein said alkyl substituents of R₅ is optionally substituted with —NH₂.

In another embodiment, R₁ is selected from phenyl, pyridinyl, furanyl, isoxazolyl, pyrazolyl and thiazolyl.

In another embodiment, said phenyl, pyridinyl and furanyl of R₁ can be optionally substituted with 1 to 3 radicals independently selected from fluoro, chloro, trifluoromethyl, methyl, ethyl, methoxy, trifluoromethoxy, difluoromethyl, 1,1-difluoroethyl, ethyl-piperazinyl-methyl, ethyl-piperazinyl; t-butyl, isopropyl, diethyl-amino-ethoxy, dimethyl-amino, 2-cyanopropan-2-yl and 1-cyanocyclopropyl.

In another embodiment, said isoxazolyl, pyrazolyl and thiazolyl of R₁ can be optionally substituted with 1 to 2 radicals independently selected from fluoro, chloro, trifluoromethyl, methyl, ethyl, methoxy, trifluoromethoxy, difluoromethyl, 1,1-difluoroethyl, t-butyl, isopropyl, dimethyl-amino, 2-cyanopropan-2-yl and 1-cyanocyclopropyl.

In another embodiment, some compounds of the invention show at least a 50% increase in bioavailability, comparing plasma concentrations, over compounds that do not contain a urea linkage. For example, the following is a comparison between compound 17 of table 1 and equivalent compound containing a non-urea linkage (compound A):

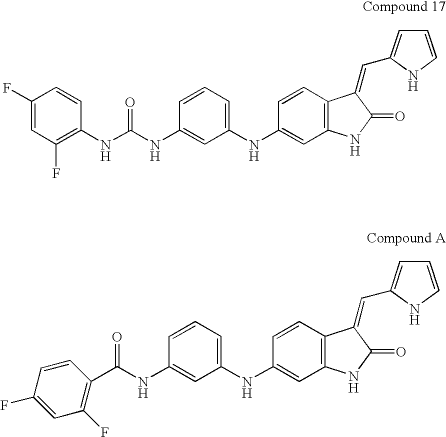

The plasma concentration of compound 17 is 2.2 times, 4.1 times, 4.7 times and 2.3 times that of compound A at 30 minutes, 1 hour, 3 hours and 5 hours, respectively. Compounds of the invention have urea linkage that offers more stability through better solubility or better permeability compared with the amide linkages.

Compounds of the invention are significantly more potent for TrkA, TrkB and TrkC compared with the equivalent compounds where the urea linkage is replaced with an amide linkage. For example, compound 17 is at least 132 fold more potent for TrkA, TrkB and TrkC than compound A.

In another embodiment are compounds detailed in the Examples and Table I, infra.

In a further embodiment are compounds selected from: 1-{3-[2-oxo-3-(1H-pyrrol-2-ylmethylene)-2,3-dihydro-1H-indol-6-ylamino]phenyl}-3-(3-trifluoromethylphenyl)urea; 1-{3-[2-oxo-3-(1H-pyrrol-2-ylmethylene)-2,3-dihydro-1H-indol-6-ylamino]-phenyl}-3-(3,4,5-trifluorophenyl)urea; 1-{3-[2-oxo-3-(1H-pyrrol-2-ylmethylene)-2,3-dihydro-1H-indol-6-ylamino]phenyl}-3-(2,4,5-trifluorophenyl)urea; 1-{3-[2-oxo-3-(1H-pyrrol-2-ylmethylene)-2,3-dihydro-1H-indol-6-ylamino]-phenyl}-3-phenyl-urea; 1-{4-Methyl-3-[2-oxo-3-(1H-pyrrol-2-ylmethylene)-2,3-dihydro-1H-indol-6-ylamino]-phenyl}-3-phenyl-urea; 1-{2-Methyl-5-[2-oxo-3-(1H-pyrrol-2-ylmethylene)-2,3-dihydro-1H-indol-6-ylamino]-phenyl}-3-phenyl-urea; 1-(2-Fluoro-phenyl)-3-{3-[2-oxo-3-(1H-pyrrol-2-ylmethylene)-2,3-dihydro-1H-indol-6-ylamino]-phenyl}-urea; 1-(3-Fluoro-phenyl)-3-{3-[2-oxo-3-(1H-pyrrol-2-ylmethylene)-2,3-dihydro-1H-indol-6-ylamino]-phenyl}-urea; 1-(4-Fluoro-phenyl)-3-{3-[2-oxo-3-(1H-pyrrol-2-ylmethylene)-2,3-dihydro-1H-indol-6-ylamino]-phenyl}-urea; 1-(2-Chloro-phenyl)-3-{3-[2-oxo-3-(1H-pyrrol-2-ylmethylene)-2,3-dihydro-1H-indol-6-ylamino]-phenyl}-urea; 1-(3-Chloro-phenyl)-3-{3-[2-oxo-3-(1H-pyrrol-2-ylmethylene)-2,3-dihydro-1H-indol-6-ylamino]-phenyl}-urea; 1-(4-Chloro-phenyl)-3-{3-[2-oxo-3-(1H-pyrrol-2-ylmethylene)-2,3-dihydro-1H-indol-6-ylamino]-phenyl}-urea; 1-(2,3-Difluoro-phenyl)-3-{3-[2-oxo-3-(1H-pyrrol-2-ylmethylene)-2,3-dihydro-1H-indol-6-ylamino]-phenyl}-urea; 1-(2,5-Difluoro-phenyl)-3-{3-[2-oxo-3-(1H-pyrrol-2-ylmethylene)-2,3-dihydro-1H-indol-6-ylamino]-phenyl}-urea; 1-(2,6-Difluoro-phenyl)-3-{3-[2-oxo-3-(1H-pyrrol-2-ylmethylene)-2,3-dihydro-1H-indol-6-ylamino]-phenyl}-urea; 1-(3,4-Difluoro-phenyl)-3-{3-[2-oxo-3-(1H-pyrrol-2-ylmethylene)-2,3-dihydro-1H-indol-6-ylamino]-phenyl}-urea; 1-(2,4-Difluoro-phenyl)-3-{3-[2-oxo-3-(1H-pyrrol-2-ylmethylene)-2,3-dihydro-1H-indol-6-ylamino]-phenyl}-urea; 1-{3-[2-oxo-3-(1H-pyrrol-2-ylmethylene)-2,3-dihydro-1H-indol-6-ylamino]-phenyl}-3-(2,4,6-trifluoro-phenyl)-urea; 1-(3,5-Difluoro-phenyl)-3-{3-[2-oxo-3-(1H-pyrrol-2-ylmethylene)-2,3-dihydro-1H-indol-6-ylamino]-phenyl}-urea; 1-{3-[2-Oxo-3-(1H-pyrrol-2-ylmethylene)-2,3-dihydro-1H-indol-6-ylamino]-phenyl}-3-(2,3,4-trifluoro-phenyl)-urea; 1-(2-Fluoro-phenyl)-3-{4-methyl-3-[2-oxo-3-(1H-pyrrol-2-ylmethylene)-2,3-dihydro-1H-indol-6-ylamino]-phenyl}-urea; 1-(3-Fluoro-phenyl)-3-{4-methyl-3-[2-oxo-3-(1H-pyrrol-2-ylmethylene)-2,3-dihydro-1H-indol-6-ylamino]-phenyl}-urea; 1-(2-Chloro-phenyl)-3-{4-methyl-3-[2-oxo-3-(1H-pyrrol-2-ylmethylene)-2,3-dihydro-1H-indol-6-ylamino]-phenyl}-urea; 1-(4-Fluoro-phenyl)-3-{4-methyl-3-[2-oxo-3-(1H-pyrrol-2-ylmethylene)-2,3-dihydro-1H-indol-6-ylamino]-phenyl}-urea; 1-(4-Chloro-phenyl)-3-{4-methyl-3-[2-oxo-3-(1H-pyrrol-2-ylmethylene)-2,3-dihydro-1H-indol-6-ylamino]-phenyl}-urea; 1-(3-Chloro-phenyl)-3-{4-methyl-3-[2-oxo-3-(1H-pyrrol-2-ylmethylene)-2,3-dihydro-1H-indol-6-ylamino]-phenyl}-urea; 1-{4-Methyl-3-[2-oxo-3-(1H-pyrrol-2-ylmethylene)-2,3-dihydro-1H-indol-6-ylamino]-phenyl}-3-(3-trifluoromethyl-phenyl)-urea; 1-(2,5-Difluoro-phenyl)-3-{4-methyl-3-[2-oxo-3-(1H-pyrrol-2-ylmethylene)-2,3-dihydro-1H-indol-6-ylamino]-phenyl}-urea; 1-(2,4-Difluoro-phenyl)-3-{4-methyl-3-[2-oxo-3-(1H-pyrrol-2-ylmethylene)-2,3-dihydro-1H-indol-6-ylamino]-phenyl}-urea; 1-(3,5-Difluoro-phenyl)-3-{4-methyl-3-[2-oxo-3-(1H-pyrrol-2-ylmethylene)-2,3-dihydro-1H-indol-6-ylamino]-phenyl}-urea; 1-(2,6-Difluoro-phenyl)-3-{4-methyl-3-[2-oxo-3-(1H-pyrrol-2-ylmethylene)-2,3-dihydro-1H-indol-6-ylamino]-phenyl}-urea; 1-(3,4-Difluoro-phenyl)-3-{4-methyl-3-[2-oxo-3-(1H-pyrrol-2-ylmethylene)-2,3-dihydro-1H-indol-6-ylamino]-phenyl}-urea; 1-{4-Methyl-3-[2-oxo-3-(1H-pyrrol-2-ylmethylene)-2,3-dihydro-1H-indol-6-ylamino]-phenyl}-3-(2,4,6-trifluoro-phenyl)-urea; 1-(3-Chloro-4-fluoro-phenyl)-3-{3-[2-oxo-3-(1H-pyrrol-2-ylmethylene)-2,3-dihydro-1H-indol-6-ylamino]-phenyl}-urea; 1-(4-Chloro-2-fluoro-phenyl)-3-{3-[2-oxo-3-(1H-pyrrol-2-ylmethylene)-2,3-dihydro-1H-indol-6-ylamino]-phenyl}-urea; 1-(4-Fluoro-3-methyl-phenyl)-3-{3-[2-oxo-3-(1H-pyrrol-2-ylmethylene)-2,3-dihydro-1H-indol-6-ylamino]-phenyl}-urea; 1-(2-Fluoro-5-methyl-phenyl)-3-{3-[2-oxo-3-(1H-pyrrol-2-ylmethylene)-2,3-dihydro-1H-indol-6-ylamino]-phenyl}-urea; 1-(3,5-Dimethyl-phenyl)-3-{3-[2-oxo-3-(1H-pyrrol-2-ylmethylene)-2,3-dihydro-1H-indol-6-ylamino]-phenyl}-urea; 1-(3-Ethyl-phenyl)-3-{3-[2-oxo-3-(1H-pyrrol-2-ylmethylene)-2,3-dihydro-1H-indol-6-ylamino]-phenyl}-urea; 1-(2-Fluoro-3-trifluoromethyl-phenyl)-3-{3-[2-oxo-3-(1H-pyrrol-2-ylmethylene)-2,3-dihydro-1H-indol-6-ylamino]-phenyl}-urea; 1-(3-Fluoro-5-trifluoromethyl-phenyl)-3-{3-[2-oxo-3-(1H-pyrrol-2-ylmethylene)-2,3-dihydro-1H-indol-6-ylamino]-phenyl}-urea; 1-(4-Fluoro-3-trifluoromethyl-phenyl)-3-{3-[2-oxo-3-(1H-pyrrol-2-ylmethylene)-2,3-dihydro-1H-indol-6-ylamino]-phenyl}-urea; 1-(2-Fluoro-5-trifluoromethyl-phenyl)-3-{3-[2-oxo-3-(1H-pyrrol-2-ylmethylene)-2,3-dihydro-1H-indol-6-ylamino]-phenyl}-urea; 1-{3-[2-Oxo-3-(1H-pyrrol-2-ylmethylene)-2,3-dihydro-1H-indol-6-ylamino]-phenyl}-3-(3-trifluoromethoxy-phenyl)-urea; 1-(3-Methoxy-phenyl)-3-{3-[2-oxo-3-(1H-pyrrol-2-ylmethylene)-2,3-dihydro-1H-indol-6-ylamino]-phenyl}-urea; 1-(3-Difluoromethyl-phenyl)-3-{3-[2-oxo-3-(1H-pyrrol-2-ylmethylene)-2,3-dihydro-1H-indol-6-ylamino]-phenyl}-urea; 1-(4-Fluoro-3-methoxy-phenyl)-3-{3-[2-oxo-3-(1H-pyrrol-2-ylmethylene)-2,3-dihydro-1H-indol-6-ylamino]-phenyl}-urea; 1-[3-(1,1-Difluoro-ethyl)-phenyl]-3-{3-[2-oxo-3-(1H-pyrrol-2-ylmethylene)-2,3-dihydro-1H-indol-6-ylamino]-phenyl}-urea; 1-(3-Chloro-4-fluoro-phenyl)-3-{4-methyl-3-[2-oxo-3-(1H-pyrrol-2-ylmethylene)-2,3-dihydro-1H-indol-6-ylamino]-phenyl}-urea; 1-(4-Chloro-2-fluoro-phenyl)-3-{4-methyl-3-[2-oxo-3-(1H-pyrrol-2-ylmethylene)-2,3-dihydro-1H-indol-6-ylamino]-phenyl}-urea; 1-(3,5-Dimethyl-phenyl)-3-{4-methyl-3-[2-oxo-3-(1H-pyrrol-2-ylmethylene)-2,3-dihydro-1H-indol-6-ylamino]-phenyl}-urea; 1-(3-Ethyl-phenyl)-3-{4-methyl-3-[2-oxo-3-(1H-pyrrol-2-ylmethylene)-2,3-dihydro-1H-indol-6-ylamino]-phenyl}-urea; 1-(3-Fluoro-5-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(4-Fluoro-3-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(2-Fluoro-5-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(3-Difluoromethyl-phenyl)-3-{4-methyl-3-[2-oxo-3-(1H-pyrrol-2-ylmethylene)-2,3-dihydro-1H-indol-6-ylamino]-phenyl}-urea; 1-(4-Fluoro-3-methoxy-phenyl)-3-{4-methyl-3-[2-oxo-3-(1H-pyrrol-2-yl methylene)-2,3-dihydro-1H-indol-6-ylamino]-phenyl}-urea; 1-[3-(1,1-Difluoro-ethyl)-phenyl]-3-{4-methyl-3-[2-oxo-3-(1H-pyrrol-2-ylmethylene)-2,3-dihydro-1H-indol-6-ylamino]-phenyl}-urea; 1-(4-Methyl-3-trifluoromethyl-phenyl)-3-{3-[2-oxo-3-(1H-pyrrol-2-ylmethylene)-2,3-dihydro-1H-indol-6-ylamino]-phenyl}-urea; 1-(4-Chloro-3-trifluoromethyl-phenyl)-3-{3-[2-oxo-3-(1H-pyrrol-2-ylmethylene)-2,3-dihydro-1H-indol-6-ylamino]-phenyl}-urea; 1-(3,5-Dichloro-phenyl)-3-{3-[2-oxo-3-(1H-pyrrol-2-ylmethylene)-2,3-dihydro-1H-indol-6-ylamino]-phenyl}-urea; 1-(2,4-Dichloro-phenyl)-3-{3-[2-oxo-3-(1H-pyrrol-2-ylmethylene)-2,3-dihydro-1H-indol-6-ylamino]-phenyl}-urea; 1-(3,4-Dichloro-phenyl)-3-{3-[2-oxo-3-(1H-pyrrol-2-ylmethylene)-2,3-dihydro-1H-indol-6-ylamino]-phenyl}-urea; 1-(2,5-Dichloro-phenyl)-3-{3-[2-oxo-3-(1H-pyrrol-2-ylmethylene)-2,3-dihydro-1H-indol-6-ylamino]-phenyl}-urea; 1-(2,6-Dichloro-phenyl)-3-{3-[2-oxo-3-(1H-pyrrol-2-ylmethylene)-2,3-dihydro-1H-indol-6-ylamino]-phenyl}-urea; 1-[4-(4-Ethyl-piperazin-1-ylmethyl)-3-trifluoromethyl-phenyl]-3-{3-[2-oxo-3-(1H-pyrrol-2-ylmethylene)-2,3-dihydro-1H-indol-6-ylamino]-phenyl}-urea; 1-[3-(4-Ethyl-piperazin-1-yl)-5-trifluoromethyl-phenyl]-3-{3-[2-oxo-3-(1H-pyrrol-2-ylmethylene)-2,3-dihydro-1H-indol-6-ylamino]-phenyl}-urea; 1-(3,5-Bis-trifluoromethyl-phenyl)-3-{3-[2-oxo-3-(1H-pyrrol-2-ylmethylene)-2,3-dihydro-1H-indol-6-ylamino]-phenyl}-urea; 1-{4-Methyl-3-[2-oxo-3-(1H-pyrrol-2-ylmethylene)-2,3-dihydro-1H-indol-6-ylamino]-phenyl}-3-(4-methyl-3-trifluoromethyl-phenyl)-urea; 1-(4-Chloro-3-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(3,5-Dichloro-phenyl)-3-{4-methyl-3-[2-oxo-3-(1H-pyrrol-2-ylmethylene)-2,3-dihydro-1H-indol-6-ylamino]-phenyl}-urea; 1-(2,5-Dichloro-phenyl)-3-{4-methyl-3-[2-oxo-3-(1H-pyrrol-2-ylmethylene)-2,3-dihydro-1H-indol-6-ylamino]-phenyl}-urea; 1-(2,4-Dichloro-phenyl)-3-{4-methyl-3-[2-oxo-3-(1H-pyrrol-2-ylmethylene)-2,3-dihydro-1H-indol-6-ylamino]-phenyl}-urea; 1-(3,4-Dichloro-phenyl)-3-{4-methyl-3-[2-oxo-3-(1H-pyrrol-2-ylmethylene)-2,3-dihydro-1H-indol-6-ylamino]-phenyl}-urea; 1-(3,5-Bis-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(2,5-Dimethyl-furan-3-yl)-3-{3-[2-oxo-3-(1H-pyrrol-2-ylmethylene)-2,3-dihydro-1H-indol-6-ylamino]-phenyl}-urea; 1-(5-tert-Butyl-2-methyl-furan-3-yl)-3-{3-[2-oxo-3-(1H-pyrrol-2-ylmethylene)-2,3-dihydro-1H-indol-6-ylamino]-phenyl}-urea; 1-{2-Methyl-5-[2-oxo-3-(1H-pyrrol-2-ylmethylene)-2,3-dihydro-1H-indol-6-ylamino]-phenyl}-3-(3-trifluoromethyl-phenyl)-urea; 1-(2-Fluoro-5-trifluoromethyl-phenyl)-3-{2-methyl-5-[2-oxo-3-(1H-pyrrol-2-ylmethylene)-2,3-dihydro-1H-indol-6-ylamino]-phenyl}-urea; 1-{4-Methoxy-3-[2-oxo-3-(1H-pyrrol-2-ylmethylene)-2,3-dihydro-1H-indol-6-ylamino]-phenyl}-3-(3-trifluoromethyl-phenyl)-urea; 1-(2-Fluoro-5-trifluoromethyl-phenyl)-3-{4-methoxy-3-[2-oxo-3-(1H-pyrrol-2-ylmethylene)-2,3-dihydro-1H-indol-6-ylamino]-phenyl}-urea; 1-{4-Fluoro-3-[2-oxo-3-(1H-pyrrol-2-ylmethylene)-2,3-dihydro-1H-indol-6-ylamino]-phenyl}-3-(2-fluoro-5-trifluoromethyl-phenyl)-urea; 1-{4-Fluoro-3-[2-oxo-3-(1H-pyrrol-2-ylmethylene)-2,3-dihydro-1H-indol-6-ylamino]-phenyl}-3-(3-trifluoromethyl-phenyl)-urea; 1-{2-Fluoro-5-[2-oxo-3-(1H-pyrrol-2-ylmethylene)-2,3-dihydro-1H-indol-6-ylamino]-phenyl}-3-(3-trifluoromethyl-phenyl)-urea; 1-{2-Fluoro-5-[2-oxo-3-(1H-pyrrol-2-ylmethylene)-2,3-dihydro-1H-indol-6-ylamino]-phenyl}-3-(2-fluoro-5-trifluoromethyl-phenyl)-urea; 1-(2-Chloro-5-trifluoromethyl-phenyl)-3-{2-methyl-5-[2-oxo-3-(1H-pyrrol-2-ylmethylene)-2,3-dihydro-1H-indol-6-ylamino]-phenyl}-urea; 1-(2-Chloro-5-trifluoromethyl-phenyl)-3-{4-methoxy-3-[2-oxo-3-(1H-pyrrol-2-ylmethylene)-2,3-dihydro-1H-indol-6-ylamino]-phenyl}-urea; 1-(2-Chloro-5-trifluoromethyl-phenyl)-3-{2-fluoro-5-[2-oxo-3-(1H-pyrrol-2-ylmethylene)-2,3-dihydro-1H-indol-6-ylamino]-phenyl}-urea; 1-{4-Methyl-3-[2-oxo-3-(1H-pyrrol-2-ylmethylene)-2,3-dihydro-1H-indol-6-ylamino]-phenyl}-3-m-tolyl-urea; 1-{3-[2-Oxo-3-(1H-pyrrol-2-ylmethylene)-2,3-dihydro-1H-indol-6-ylamino]-phenyl}-3-m-tolyl-urea; 1-(3-Isopropyl-phenyl)-3-{3-[2-oxo-3-(1H-pyrrol-2-ylmethylene)-2,3-dihydro-1H-indol-6-ylamino]-phenyl}-urea; 1-(3-tert-Butyl-phenyl)-3-{3-[2-oxo-3-(1H-pyrrol-2-ylmethylene)-2,3-dihydro-1H-indol-6-ylamino]-phenyl}-urea; 1-{3-Cyano-5-[2-oxo-3-(1H-pyrrol-2-ylmethylene)-2,3-dihydro-1H-indol-6-ylamino]-phenyl}-3-(2-fluoro-5-trifluoromethyl-phenyl)-urea; 1-(3-Fluoro-5-trifluoromethyl-phenyl)-3-{3-[2-oxo-3-(1H-pyrrol-2-ylmethylene)-2,3-dihydro-1H-indol-6-ylsulfanyl]-phenyl}-urea; 1-(2-Fluoro-5-trifluoromethyl-phenyl)-3-{3-[2-oxo-3-(1H-pyrrol-2-ylmethylene)-2,3-dihydro-1H-indol-6-ylsulfanyl]-phenyl}-urea; 1-{3-Cyano-5-[2-oxo-3-(1H-pyrrol-2-ylmethylene)-2,3-dihydro-1H-indol-6-ylamino]-phenyl}-3-(3-trifluoromethyl-phenyl)-urea; 1-{3-[2-Oxo-3-(1H-pyrrol-2-ylmethylene)-2,3-dihydro-1H-indol-6-ylsulfanyl]-phenyl}-3-(3-trifluoromethyl-phenyl)-urea; 1-{3-Cyano-5-[2-oxo-3-(1H-pyrrol-2-ylmethylene)-2,3-dihydro-1H-indol-6-ylamino]-phenyl}-3-(3-fluoro-5-trifluoromethyl-phenyl)-urea; 1-(2-Chloro-5-trifluoromethyl-phenyl)-3-{4-fluoro-3-[2-oxo-3-(1H-pyrrol-2-ylmethylene)-2,3-dihydro-1H-indol-6-ylamino]-phenyl}-urea; 1-{4-Fluoro-3-[2-oxo-3-(1H-pyrrol-2-ylmethylene)-2,3-dihydro-1H-indol-6-ylamino]-phenyl}-3-(3-trifluoromethoxy-phenyl)-urea; 1-{2-Fluoro-5-[2-oxo-3-(1H-pyrrol-2-ylmethylene)-2,3-dihydro-1H-indol-6-ylamino]-phenyl}-3-(3-trifluoromethoxy-phenyl)-urea; 1-(3-Chloro-phenyl)-3-{3-[3-(5-methyl-3H-imidazol-4-ylmethylene)-2-oxo-2,3-dihydro-1H-indol-6-ylamino]-phenyl}-urea; 1-{3-[2-Oxo-3-(1H-pyrrol-2-ylmethylene)-2,3-dihydro-1H-indol-5-ylamino]-phenyl}-3-phenyl-urea; 1-{3-[2-oxo-3-(1H-pyrrol-2-ylmethylene)-2,3-dihydro-1H-indol-6-ylamino]-phenyl}-3-(2-trifluoromethyl-phenyl)-urea; 1-{3-[2-oxo-3-(1H-pyrrol-2-ylmethylene)-2,3-dihydro-1H-indol-6-ylamino]-phenyl}-3-(4-trifluoromethyl-phenyl)-urea; 1-{3-[3-(4-Cyano-1H-pyrrol-2-ylmethylene)-2-oxo-2,3-dihydro-1H-indol-6-ylamino]-phenyl}-3-phenyl-urea; 1-(4-Fluoro-phenyl)-3-{3-[3-(5-methyl-3H-imidazol-4-ylmethylene)-2-oxo-2,3-dihydro-1H-indol-6-ylamino]-phenyl}-urea; 5-(6-{3-[3-(3-Fluoro-phenyl)-ureido]-phenylamino}-2-oxo-1,2-dihydro-indol-3-ylidenemethyl)-1H-pyrrole-3-carboxylic acid; 5-(6-{3-[3-(3-Fluoro-phenyl)-ureido]-phenylamino}-2-oxo-1,2-dihydro-indol-3-ylidenemethyl)-1H-pyrrole-3-carboxylic acid (2-diethylamino-ethyl)-amide; 1-(4-Dimethylamino-phenyl)-3-{3-[2-oxo-3-(1H-pyrrol-2-ylmethylene)-2,3-dihydro-1H-indol-6-ylamino]-phenyl}-urea; 1-(3-Fluoro-phenyl)-3-{3-[3-(4-methyl-1H-imidazol-2-ylmethylene)-2-oxo-2,3-dihydro-1H-indol-6-ylamino]-phenyl}-urea; 5-(6-{3-[3-(4-Fluoro-phenyl)-ureido]-phenylamino}-2-oxo-1,2-dihydro-indol-3-ylidenemethyl)-1H-pyrrole-3-carboxylic acid; 1-{3-Methoxy-5-[2-oxo-3-(1H-pyrrol-2-ylmethylene)-2,3-dihydro-1H-indol-6-ylamino]-phenyl}-3-phenyl-urea; 1-(3-Chloro-phenyl)-3-{3-[3-(4-methyl-1H-imidazol-2-ylmethylene)-2-oxo-2,3-dihydro-1H-indol-6-ylamino]-phenyl}-urea; 1-(3-Chloro-phenyl)-3-{3-[3-(2-ethyl-5-methyl-3H-imidazol-4-ylmethylene)-2-oxo-2,3-dihydro-1H-indol-6-ylamino]-phenyl}-urea; 1-(2,6-Dichloro-pyridin-4-yl)-3-{3-[2-oxo-3-(1H-pyrrol-2-ylmethylene)-2,3-dihydro-1H-indol-6-ylamino]-phenyl}-urea; 1-(5-Methyl-2-trifluoromethyl-furan-3-yl)-3-{3-[2-oxo-3-(1H-pyrrol-2-ylmethylene)-2,3-dihydro-1H-indol-6-ylamino]-phenyl}-urea; 1-{3-[2-oxo-3-(1H-pyrrol-2-ylmethylene)-2,3-dihydro-1H-indol-6-ylamino]-phenyl}-3-pyridin-3-yl-urea; 5-(2-Oxo-6-{3-[3-(3-trifluoromethyl-phenyl)-ureido]-phenylamino}-1,2-dihydro-indol-3-ylidenemethyl)-1H-pyrrole-3-carboxylic acid; 5-(2-Oxo-6-{3-[3-(3-trifluoromethyl-phenyl)-ureido]-phenylamino}-1,2-dihydro-indol-3-ylidenemethyl)-1H-pyrrole-3-carboxylic acid methyl ester; 1-{3-[3-(4-Methyl-1H-imidazol-2-ylmethylene)-2-oxo-2,3-dihydro-1H-indol-6-ylamino]-phenyl}-3-(3-trifluoromethyl-phenyl)-urea; 1-{3-[3-(2-Ethyl-5-methyl-3H-imidazol-4-ylmethylene)-2-oxo-2,3-dihydro-1H-indol-6-ylamino]-phenyl}-3-(3-trifluoromethyl-phenyl)-urea; 5-(6-{2-Methyl-5-[3-(3-trifluoromethyl-phenyl)-ureido]-phenylamino}-2-oxo-1,2-dihydro-indol-3-ylidenemethyl)-1H-pyrrole-3-carboxylic acid methyl ester; 1-{3-[2-oxo-3-(1H-pyrrol-2-ylmethylene)-2,3-dihydro-1H-indol-6-ylamino]-phenyl}-3-(4-trifluoromethoxy-phenyl)-urea; 1-{3-[3-(4-Cyano-1H-pyrrol-2-ylmethylene)-2-oxo-2,3-dihydro-1H-indol-6-ylamino]-phenyl}-3-(3-trifluoromethyl-phenyl)-urea; 1-{3-[3-(4-Cyano-1H-pyrrol-2-ylmethylene)-2-oxo-2,3-dihydro-1H-indol-6-ylamino]-4-methyl-phenyl}-3-(3-trifluoromethyl-phenyl)-urea; 1-{3-[2-oxo-3-(1H-pyrrol-2-ylmethylene)-2,3-dihydro-1H-indol-6-ylamino]-phenyl}-3-(3-trifluoromethyl-benzyl)-urea; 5-(6-{3-[3-(5-Methyl-2-trifluoromethyl-furan-3-yl)-ureido]-phenylamino}-2-oxo-1,2-dihydro-indol-3-ylidenemethyl)-1H-pyrrole-3-carboxylic acid methyl ester; 1-[3-(Cyano-dimethyl-methyl)-phenyl]-3-{3-[2-oxo-3-(1H-pyrrol-2-ylmethylene)-2,3-dihydro-1H-indol-6-ylamino]-phenyl}-urea; 1-[3-(1-Cyano-cyclopropyl)-phenyl]-3-{3-[2-oxo-3-(1H-pyrrol-2-ylmethylene)-2,3-dihydro-1H-indol-6-ylamino]-phenyl}-urea; 1-{3-Fluoro-5-[2-oxo-3-(1H-pyrrol-2-ylmethylene)-2,3-dihydro-1H-indol-6-ylamino]-phenyl}-3-(3-trifluoromethyl-phenyl)-urea; and 1-(2,5-Dichloro-phenyl)-3-{4-fluoro-3-[2-oxo-3-(1H-pyrrol-2-ylmethylene)-2,3-dihydro-1H-indol-6-ylamino]-phenyl}-urea.

Another embodiment includes all suitable isotopic variations of the compounds of the invention, or pharmaceutically acceptable salts thereof. An isotopic variation of a compound of the invention or a pharmaceutically acceptable salt thereof is defined as one in which at least one atom is replaced by an atom having the same atomic number but an atomic mass different from the atomic mass usually found in nature. Examples of isotopes that may be incorporated into the compounds of the invention and pharmaceutically acceptable salts thereof include but are not limited to isotopes of hydrogen, carbon, nitrogen and oxygen such as ²H, ³H, ¹³C, ¹⁴C, ¹⁵N, ¹⁷O, ¹⁸O, ³⁵S, ¹⁸F, and ³⁶Cl. Certain isotopic variations of the compounds of the invention and pharmaceutically acceptable salts thereof, for example, those in which a radioactive isotope such as ³H or ¹⁴C is incorporated, are useful in drug and/or substrate tissue distribution studies. In particular examples, ³H and ¹⁴C isotopes may be used for their ease of preparation and detectability. In other examples, substitution with isotopes such as ²H may afford certain therapeutic advantages resulting from greater metabolic stability, such as increased in vivo half-life or reduced dosage requirements. Isotopic variations of the compounds of the invention or pharmaceutically acceptable salts thereof can generally be prepared by conventional procedures using appropriate isotopic variations of suitable reagents.

### Pharmacology and Utility

Compounds of the invention modulate the activity of kinases and, as such, are useful for treating diseases or disorders in which kinases, contribute to the pathology and/or symptomology of the disease. Examples of kinases that are inhibited by the compounds and compositions described herein and against which the methods described herein are useful include, but are not limited to, TrkA, TrkB, TrkC, PDGFR and c-kit kinases.

The trk families of neurotrophin receptors (TrkA or “NTKR1”, TrkB or “NTKR2”, and TrkC or “NTKR3”) are able to control tumor cell growth and survival as well as differentiation, migration and metastasis.

NTKR2 (TrkB) protein is expressed in neuroendocrine-type cells in the small intestine and colon, in the alpha cells of the pancreas, in the monocytes and macrophages of the lymph nodes and of the spleen, and in the granular layers of the epidermis. Expression of the TrkB protein has been associated with an unfavorable progression of Wilms tumors and of neuroblastomas. TkrB is, moreover, expressed in cancerous prostate cells but not in normal cells.

NTRK3 (TrkC) and its closely related family members NTRK1 (TrkA) and NTRK2 (TrkB) are implicated in the development and progression of cancer, possibly by upregulation of either the receptor, their ligand (Nerve Growth Factor, Brain Derived Neurotrophic Factor, Neurotrophins) or both. High expression of NTRK2 and/or its ligand BDNF has been shown in pancreatic and prostate carcinomas, Wilm's tumors and neuroblastomas. In addition, high expression of NTRK3 is a hallmark of Melanoma, especially in cases with brain metastasis. In many cases high Trk expression is associated with aggressive tumor behavior, poor prognosis and metastasis.

NTRK2 is a potent inhibitor of anoikis, defined as apoptosis induced by loss of attachment of a cell to its matrix. By activating the Phosphatidylinositol-3-kinase/Protein Kinase B signaling axis, NTRK2 was shown to promote the survival of non-transformed epithelial cells in 3-dimensional cultures and to induce tumor formation and metastasis of those cells in immuno-compromised mice.

Genetic abnormalities, i.e. point mutations and chromosomal rearrangements involving both NTRK2 and NTRK3 have been found in a variety of cancer types. In a kinome-wide approach to identify point mutants in tyrosine kinases both NTRK2 and NTRK3 mutations were found in cell lines and primary samples from patients with colorectal cancer (Manning et al., 2002, Bardelli et al., 2003). Although no further validation of the various mutants was presented in this analysis, the implication of Trk family members in regulating metastasis suggests a functional relevance of this observation in colorectal cancer.

In addition, chromosomal translocations involving both NTRK1 and NTRK3 have been found in several different types of tumors. Gene rearrangements involving NTRK1 and a set of different fusion partners (TPM3, TPR, TFG) are a hallmark of a subset of papillary thyroid cancers. Moreover, secretary breast cancer, infant fibrosarcoma and congenital mesoblastic nephroma have been shown to be associated with a chromosomal rearrangement t(12;15) generating a ETV6-NTRK3 fusion gene that was shown to have constitutive kinase activity and transforming potential in several different cell lines including fibroblasts, hematopoietic cells and breast epithelial cells.

PDGF (Platelet-derived Growth Factor) is a very commonly occurring growth factor, which plays an important role both in normal growth and also in pathological cell proliferation, such as is seen in carcinogenesis and in diseases of the smooth-muscle cells of blood vessels, for example in atherosclerosis and thrombosis. Compounds of the invention can inhibit PDGF receptor (PDGFR) activity and are, therefore, suitable for the treatment of tumor diseases, such as gliomas, sarcomas, prostate tumors, and tumors of the colon, breast, and ovary.

Compounds of the present invention, can be used not only as a tumor-inhibiting substance, for example in small cell lung cancer, but also as an agent to treat non-malignant proliferative disorders, such as atherosclerosis, thrombosis, psoriasis, scleroderma and fibrosis, as well as for the protection of stem cells, for example to combat the hemotoxic effect of chemotherapeutic agents, such as 5-fluoruracil, and in asthma. Compounds of the invention can especially be used for the treatment of diseases, which respond to an inhibition of the PDGF receptor kinase.

Compounds of the present invention show useful effects in the treatment of disorders arising as a result of transplantation, for example, allogenic transplantation, especially tissue rejection, such as especially obliterative bronchiolitis (OB), i.e. a chronic rejection of allogenic lung transplants. In contrast to patients without OB, those with OB often show an elevated PDGF concentration in bronchoalveolar lavage fluids.

Compounds of the present invention are also effective in diseases associated with vascular smooth-muscle cell migration and proliferation (where PDGF and PDGF-R often also play a role), such as restenosis and atherosclerosis. These effects and the consequences thereof for the proliferation or migration of vascular smooth-muscle cells in vitro and in vivo can be demonstrated by administration of the compounds of the present invention, and also by investigating its effect on the thickening of the vascular intima following mechanical injury in vivo.

Abelson tyrosine kinase (i.e. Abl, c-Abl) is involved in the regulation of the cell cycle, in the cellular response to genotoxic stress, and in the transmission of information about the cellular environment through integrin signaling. Overall, it appears that the Abl protein serves a complex role as a cellular module that integrates signals from various extracellular and intracellular sources and that influences decisions in regard to cell cycle and apoptosis. Abelson tyrosine kinase includes sub-types derivatives such as the chimeric fusion (oncoprotein) BCR-Abl with deregulated tyrosine kinase activity or the v-Abl. BCR-Abl is critical in the pathogenesis of 95% of chronic myelogenous leukemia (CML) and 10% of acute lymphocytic leukemia. STI-571 (Gleevec) is an inhibitor of the oncogenic BCR-Abl tyrosine kinase and is used for the treatment of chronic myeloid leukemia (CML). However, some patients in the blast crisis stage of CML are resistant to STI-571 due to mutations in the BCR-Abl kinase. Over 22 mutations have been reported to date with the most common being G250E, E255V, T315I, F317L and M351T.

Compounds of the present invention inhibit abl kinase, especially v-abl kinase. The compounds of the present invention also inhibit wild-type BCR-Abl kinase and mutations of BCR-Abl kinase and are thus suitable for the treatment of Bcr-abl-positive cancer and tumor diseases, such as leukemias (especially chronic myeloid leukemia and acute lymphoblastic leukemia, where especially apoptotic mechanisms of action are found), and also shows effects on the subgroup of leukemic stem cells as well as potential for the purification of these cells in vitro after removal of said cells (for example, bone marrow removal) and reimplantation of the cells once they have been cleared of cancer cells (for example, reimplantation of purified bone marrow cells).

Certain abnormal proliferative conditions are believed to be associated with raf expression and are, therefore, believed to be responsive to inhibition of raf expression. Abnormally high levels of expression of the raf protein are also implicated in transformation and abnormal cell proliferation. These abnormal proliferative conditions are also believed to be responsive to inhibition of raf expression. For example, expression of the c-raf protein is believed to play a role in abnormal cell proliferation since it has been reported that 60% of all lung carcinoma cell lines express unusually high levels of c-raf mRNA and protein. Further examples of abnormal proliferative conditions are hyper-proliferative disorders such as cancers, tumors, hyperplasia, pulmonary fibrosis, angiogenesis, psoriasis, atherosclerosis and smooth muscle cell proliferation in the blood vessels, such as stenosis or restenosis following angioplasty. The cellular signaling pathway of which raf is a part has also been implicated in inflammatory disorders characterized by T-cell proliferation (T-cell activation and growth), such as tissue graft rejection, endotoxin shock, and glomerular nephritis, for example.

The family of human ribosomal S6 protein kinases consists of at least 8 members (RSK1, RSK2, RSK3, RSK4, MSK1, MSK2, p70S6K and p70S6 Kb). Ribosomal protein S6 protein kinases play important pleotropic functions, among them is a key role in the regulation of mRNA translation during protein biosynthesis (Eur. J. Biochem 2000 November; 267(21): 6321-30, Exp Cell Res. Nov. 25, 1999; 253 (1):100-9, Mol Cell Endocrinol. May 25, 1999; 151(1-2):65-77). The phosphorylation of the S6 ribosomal protein by p70S6 has also been implicated in the regulation of cell motility (Immunol. Cell Biol. 2000 August; 78(4):447-51) and cell growth (Prog. Nucleic Acid Res. Mol. Biol., 2000; 65:101-27), and hence, may be important in tumor metastasis, the immune response and tissue repair as well as other disease conditions.

Flt3 is a member of the type III receptor tyrosine kinase (RTK) family. Flt3 (fms-like tyrosine kinase) is also known as FLk-2 (fetal liver kinase 2). Aberrant expression of the Flt3 gene has been documented in both adult and childhood leukemias including acute myeloid leukemia (AML), AML with trilineage myelodysplasia (AMIJTMDS), acute lymphoblastic leukemia (ALL), and myelodysplastic syndrome (MDS). Activating mutations of the Flt3 receptor have been found in about 35% of patients with acute myeloblastic leukemia (AML), and are associated with a poor prognosis. The most common mutation involves in-frame duplication within the juxtamembrane domain, with an additional 5-10% of patients having a point mutation at asparagine 835. Both of these mutations are associated with constitutive activation of the tyrosine kinase activity of Flt3, and result in proliferation and viability signals in the absence of ligand. Patients expressing the mutant form of the receptor have been shown to have a decreased chance for cure. Thus, there is accumulating evidence for a role for hyper-activated (mutated) Flt3 kinase activity in human leukemias and myelodysplastic syndrome.

The compounds of the present invention also inhibit cellular processes involving stem-cell factor (SCF, also known as the c-kit ligand or steel factor), such as inhibiting SCF receptor (kit) autophosphorylation and SCF-stimulated activation of MAPK kinase (mitogen-activated protein kinase). MO7e cells are a human promegakaryocytic leukemia cell line, which depends on SCF for proliferation. Compounds of the invention can inhibit the autophosphorylation of SCF receptors.

Aurora-2 is a serine/threonine protein kinase that has been implicated in human cancer, such as colon, breast and other solid tumors. This kinase is believed to be involved in protein phosphorylation events that regulate the cell cycle. Specifically, Aurora-2 may play a role in controlling the accurate segregation of chromosomes during mitosis. Misregulation of the cell cycle can lead to cellular proliferation and other abnormalities. In human colon cancer tissue, the aurora-2 protein has been found to be overexpressed.

The Aurora family of serine/threonine kinases [Aurora-A (“1”), B (“2”) and C (“3”)] is essential for cell proliferation. These proteins are responsible for chromosome segregation, mitotic spindle function and cytokinesis and are linked to tumorigenesis. Elevated levels of all Aurora family members are observed in a wide variety of tumour cell lines. Aurora kinases are over-expressed in many human tumors and this is reported to be associated with chromosomal instability in mammary tumors. For example, aberrant activity of aurora A kinase has been implicated in colorectal, gastric, human bladder and ovarian cancers and high levels of Aurora-A have also been reported in renal, cervical, neuroblastoma, melanoma, lymphoma, pancreatic and prostate tumour cell lines. Aurora-B is also highly expressed in multiple human tumour cell lines, for example, leukemic cells and colorectal cancers. Aurora-C, which is normally only found in germ cells, is also over-expressed in a high percentage of primary colorectal cancers and in a variety of tumour cell lines including cervical adenocarcinoma and breast carcinoma cells. Based on the known function of the Aurora kinases, inhibition of their activity should disrupt mitosis leading to cell cycle arrest. In vivo, an Aurora inhibitor therefore slows tumor growth and induces regression.

The inactivation of Chk1 and Chk2 abrogates the G2/M arrest which is induced by damaged DNA and sensitizes the resulting checkpoint deficient cells to the killing by DNA damaging events. As cancer cells are more sensitive towards the abrogation of the G2/M checkpoint than normal cells there is great interest in compounds, which inhibit Chk1, Chk2 or Chk1 and Chk2, as a result abrogate the G2/M checkpoint and improve the killing of cancer cells by DNA damaging events.

It is believed that a wide variety of disease states and conditions can be mediated by modulating the activity of Mammalian Sterile 20-like Kinase, “Mst 1” and “Mst 2” or combinations thereof, to treat or prevent diseases which include osteoporosis, osteopenia, Paget's disease, vascular restenosis, diabetic retinopathy, macular degeneration, angiogenesis, atherosclerosis, inflammation and tumor growth.

The kinases known as PKA or cyclic AMP-dependent protein kinase, PKB or Akt, and PKC, all play key roles in signal transduction pathways responsible for oncogenesis. Compounds capable of inhibiting the activity of these kinases can be useful in the treatment of diseases characterized by abnormal cellular proliferation, such as cancer.

Rho kinase (Rock-II) participates in vasoconstriction, platelet aggregation, bronchial smooth muscle constriction, vascular smooth muscle proliferation, endothelial proliferation, stress fiber formation, cardiac hypertrophy, Na/H exchange transport system activation, adducing activation, ocular hypertension, erectile dysfunction, premature birth, retinopathy, inflammation, immune diseases, AIDS, fertilization and implantation of fertilized ovum, osteoporosis, brain functional disorder, infection of digestive tracts with bacteria, and the like.

Axl is a receptor tyrosine kinase associated with a number of disease states such as leukemia and various other cancers including gastric cancer.

Bruton's tyrosine kinase (Btk) is important for B lymphocyte development. The Btk family of non-receptor tyrosine kinases includes Btk/Atk, Itk/Emt/Tsk, Bmx/Etk, and Tec. Btk family kinases play central but diverse modulatory roles in various cellular processes. They participate in signal transduction in response to extracellular stimuli resulting in cell growth, differentiation and apoptosis. The aberrant activity of this family of kinases is linked to immunodeficiency diseases and various cancers.

Fibroblast growth factor receptor 3 was shown to exert a negative regulatory effect on bone growth and an inhibition of chondrocyte proliferation. Thanatophoric dysplasia is caused by different mutations in fibroblast growth factor receptor 3, and one mutation, TDII FGFR3, has a constitutive tyrosine kinase activity which activates the transcription factor Stat1, leading to expression of a cell-cycle inhibitor, growth arrest and abnormal bone development (Su et al., Nature, 1997, 386, 288-292). FGFR3 is also often expressed in multiple myeloma-type cancers.

Lin et al (1997) J. Clin. Invest. 100, 8: 2072-2078 and P. Lin (1998) PNAS 95, 8829-8834, have shown an inhibition of tumor growth and vascularization and also a decrease in lung metastases during adenoviral infections or during injections of the extracellular domain of Tie-2 (Tek) in breast tumor and melanoma xenograft models. Tie2 inhibitors can be used in situations where neovascularization takes place inappropriately (i.e. in diabetic retinopathy, chronic inflammation, psoriasis, Kaposi's sarcoma, chronic neovascularization due to macular degeneration, rheumatoid arthritis, infantile haemangioma and cancers).

The kinase, c-Src transmits oncogenic signals of many receptors. For example, over-expression of EGFR or HER2/neu in tumors leads to the constitutive activation of c-src, which is characteristic for the malignant cell but absent from the normal cell. On the other hand, mice deficient in the expression of c-src exhibit an osteopetrotic phenotype, indicating a key participation of c-src in osteoclast function and a possible involvement in related disorders.

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with other immunomodulatory or anti-inflammatory substances, for example when used in combination with cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, wherein R₄ is a 2-vinyl-1H-pyrrolyl derivative, can be prepared by proceeding as in the following Reaction Scheme I:

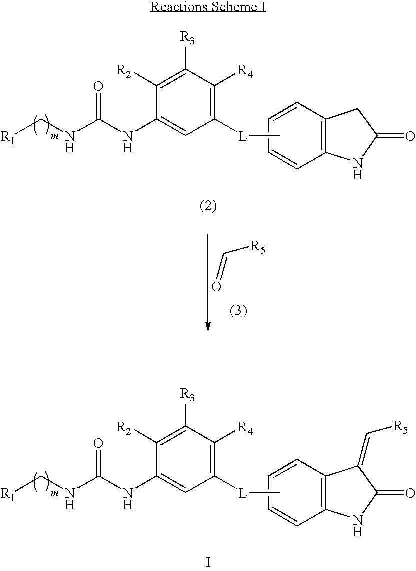

in which L, m, R₁, R₂, R₃, R₄ and R₅ are as defined for Formula I in the Summary of the Invention. A compound of Formula I can be prepared by reacting a compound of formula 2 with a compound of formula 3 in the presence of a suitable base (e.g., piperidine, or the like) and a suitable solvent (e.g., ethanol, or the like). The reaction proceeds in a temperature range of about 50 to about 120° C. and can take up to about 10 hours to complete.

Detailed examples of the synthesis of a compound of Formula I can be found in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed, during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction scheme I; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following examples that illustrate the preparation of compounds of Formula I according to the invention.

### Example 1

### 1-{3-[2-oxo-3-(1H-pyrrol-2-ylmethylene)-2,3-dihydro-1H-indol-6-ylamino]phenyl}-3-(3-trifluoromethylphenyl)urea

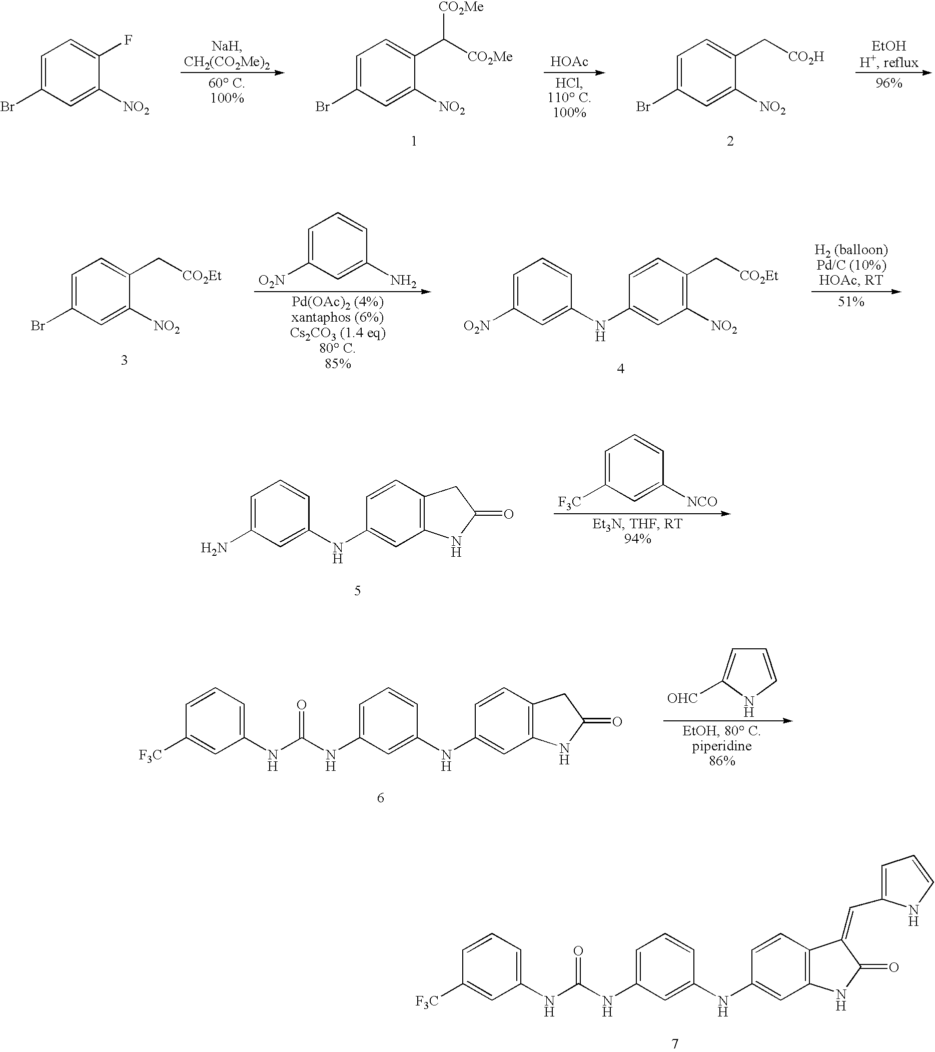

### Synthesis of 2-(4-bromo-2-nitrophenyl)malonic acid dimethyl ester (1)

To a solution of dimethyl malonate (37 g, 0.284 mol) in DMSO (100 mL) is added NaH (60% in mineral oil, 11.3 g, 0.284 mol) in portions at room temperature. The mixture is warmed to 60° C. for 15 min and then cooled to room temperature. 4-Bromo-1-fluoro-2-nitrobenzene (20.8 g, 0.0945 mol) is added drop wise to the above solution. The resulting mixture is stirred at 60° C. overnight (about 14 hr). After cooling to room temperature, the reaction is quenched by aqueous saturated NH₄Cl (100 mL). The mixture is extracted with EtOAc (3×150 mL). The combined organic layer is dried over Na₂SO₄, filtered and concentrated to oil (57.2 g, presumably containing DMSO and dimethyl malonate). LC-MS (m/z) 332.0 (M⁺), 334.0 (M⁺+2). The crude is used in the next step without further purification.

### Synthesis of (4-bromo-2-nitrophenyl)acetic acid (2)

2-(4-bromo-2-nitrophenyl)malonic acid dimethyl ester (crude, 57.2 g, 0.0945 mol, theoretical) is dissolved in 6N HCl (118 mL, 0.709 mol) and acetic acid (120 mL). The solution is heated at 110° C. overnight. The mixture is cooled to room temperature and all the solvent is removed. The resulting crude (solid, 37 g) is used in the next step without further purification. ¹H NMR (400 MHz, DMSO-d₆) δ 8.26 (d, 1H), 7.94 (dd, 1H), 7.52 (d, 1H), 3.98 (s, 2H); LC-MS (m/z) 242.0 (M⁺−17), 244.0 (M⁺+2-17).

### Synthesis of (4-bromo-2-nitrophenyl)acetic acid ethyl ester (3)

To a solution of (4-bromo-2-nitrophenyl)acetic acid (crude, 37 g) in EtOH (150 mL) is added 0.5 mL of conc. H₂SO₄. The mixture is heated at 80° C. overnight, and then cooled to room temperature. All the solvent is removed. The crude is purified by column chromatography (silica gel, EtOAc/Hexane, 1:4) to yield 26.2 g of the desired product. LC-MS (m/z) 288.0 (M⁺), 290.0 (M⁺+2).

### Synthesis of [2-nitro-4-(3-nitrophenylamino)phenyl]acetic acid ethyl ester (4)

To a flask are charged 3-nitroaniline (6.9 g, 50 mmol), (4-bromo-2-nitrophenyl)acetic acid ethyl ester (14.4 g, 50 mmol), xantaphos (868 mg, 1.5 mmol), Pd(OAc)₂ (225 mg, 1 mmol), Cs₂CO₃ (23 g, 70 mmol) and 1,4-dioxane (100 mL). The mixture is heated at 110° C. overnight. It is cooled to room temperature and filtered through Celite. The filtrate is concentrated and purified by column chromatography (ISCO, gradient, 0-100% EtOAc/hexane) to give the desired product. LC-MS (m/z) 346.1 (M⁺+1).

### Synthesis of 6-(3-aminophenylamino)-1,3-dihydroindol-2-one (5)

To a solution of [2-nitro-4-(3-nitrophenylamino)phenyl]acetic acid ethyl ester (14.6 g, 0.042 mol) in acetic acid (250 mL) is added 10% Pd/C (10 wt %, 1.46 g). The mixture is stirred under a hydrogen balloon at room temperature overnight. The mixture is filtered through Celite. The filtrate is concentrated and purified (silica gel, EtOAc/hexane, gradient, 0-100%) to give the desired product. ¹H NMR (400 MHz, DMSO-d₆) δ 10.18 (s, 1H), 7.76 (s, 1H), 6.98 (d, 1H), 6.85 (t, 1H), 6.58 (d, 1H), 6.55 (m, 1H), 6.35 (m, 1H), 6.25-6.20 (m, 1H), 6.10-6.05 (m, 1H), 4.91 (s, 2H), 3.33 (s, 2H); LC-MS (m/z) 240.1 (M⁺+1).

### Synthesis of 1-[3-(2-oxo-2,3-dihydro-1H-indol-6-ylamino)phenyl]-3-(3-trifluoro-methylphenyl)urea (6)

To a solution of 6-(3-aminophenylamino)-1,3-dihydroindol-2-one (800 mg, 3.34 mmol) in THF (35 mL) are added 3-(trifluoromethyl)phenyl isocyanate (506 μL, 3.67 mmol) and triethyl amine (1.4 mL, 10 mmol). The mixture is stirred at room temperature for 3 hr. It is then concentrated and purified by column chramatography (EtOAc/hexane, gradient, 0-100%) to give the desired product. LC-MS (m/z) 427.1 (M⁺+1).

### Synthesis of 1-{3-[2-oxo-3-(1H-pyrrol-2-ylmethylene)-2,3-dihydro-1H-indol-6-ylamino]phenyl}-3-(3-trifluoromethylphenyl)urea (7)

To a suspension of 1-[3-(2-oxo-2,3-dihydro-1H-indol-6-ylamino)phenyl]-3-(3-trifluoromethyl-phenyl)urea (1.1 g, 2.58 mmol) in ethanol (50 mL) is added pyrrole-2-carboxaldehyde (294 mg, 3.1 mmol) and piperidine (2.55 mL, 2.58 mmol). The reaction is heated at reflux for 2 hr. The suspension became clear, and then some solid started to precipitate out. The reaction mixture is cooled to room temperature. The solid is filtered out and ished with cold EtOH several times to give 940 mg of the desired product. The filtrate is concentrated and purified by column chromatography to give additional 180 mg of the desired product. M.pt. 253-256° C.; ¹H NMR (400 MHz, DMSO-d₆) δ 13.16 (s, 1H), 10.75 (s, 1H), 8.98 (s, 1H), 8.75 (s, 1H), 8.35 (s, 1H), 8.01 (s, 1H), 7.55-7.45 (m, 4H), 7.40-7.35 (m, 1H), 7.35-7.20 (m, 2H), 7.15 (t, 1H), 6.95-6.85 (m, 1H), 6.75-6.65 (m, 3H), 6.65-6.60 (m, 1H), 6.35-6.25 (m, 1H); LC-MS (m/z) 504.2 (M⁺+1).

### Example 2

### 1-{3-[2-oxo-3-(1H-pyrrol-2-ylmethylene)-2,3-dihydro-1H-indol-6-ylamino]-phenyl}-3-(3,4,5-trifluorophenyl)urea

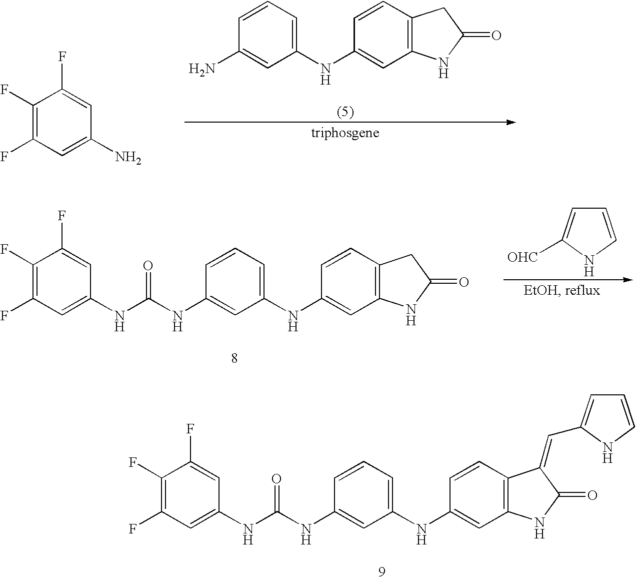

### Synthesis of 1-[3-(2-oxo-2,3-dihydro-1H-indol-6-ylamino)phenyl]-3-(3,4,5-trifluoro-phenyl)urea (8)

A solution of 3,4,5-trifluoroaniline (14.7 mg, 0.1 mmol) and diisopropylethylamine (38 μL, 0.22 mmol) in CH₂Cl₂ (2 mL) is added drop wise to a solution of triphosgene (11 mg, 0.37 mmol) in CH₂Cl₂ (1 mL) under N₂. The mixture is stirred at room temperature for 15 min. This solution is added drop wise to a solution of 6-(3-aminophenylamino)-1,3-dihydroindol-2-one (5, 20 mg, 0.084 mmol) and diisopropylethylamine (32 μL, 0.18 mmol) in CH₂Cl₂ (2 mL) over 2 min. The mixture is stirred at room temperature for 30 min. After solvent removal in vacuo, the crude product is used in the next step without further purification. LC-MS (m/z) 413.1 [M⁺+1].

### Synthesis of 1-{3-[2-oxo-3-(1H-pyrrol-2-ylmethylene)-2,3-dihydro-1H-indol-6-ylamino]-phenyl}-3-(3,4,5-trifluorophenyl)urea (9)

1-[3-(2-oxo-2,3-dihydro-1H-indol-6-ylamino)phenyl]-3-(3,4,5-trifluorophenyl)-urea (crude, 0.1 mmol, theoretical) is mixed with pyrrole-2-carboxaldehyde (9.5 mg, 0.1 mmol) and piperidine (16 μL, 0.17 mmol) in absolute ethanol (3 mL). The mixture is heated at reflux for 1 hr. After cooling to room temperature and removing solvent in vacuo, the crude product is purified using flash chromatography (hexane:ethyl acetate=1:1) to give the title compound as solid. ¹H NMR (400 MHz, DMSO-d₆) δ 10.72 (s, 1H), 8.93 (s, 1H), 8.78 (s, 1H), 8.31 (s, 1H), 7.47 (s, 1H), 7.46 (d, 1H), 7.38 (d, 1H), 7.36 (d, 1H), 7.32 (t, 1H), 7.25 (q, 1H), 7.15 (t, 1H), 6.88 (dd, 1H), 6.75-6.71 (m, 3H), 6.65 (d, 1H), 6.30 (dt, 1H); LC-MS (m/z) 490.1 [M⁺+1].

### Example 3

### 1-{3-[2-oxo-3-(1H-pyrrol-2-ylmethylene)-2,3-dihydro-1H-indol-6-ylamino]phenyl}-3-(2,4,5-trifluorophenyl)urea

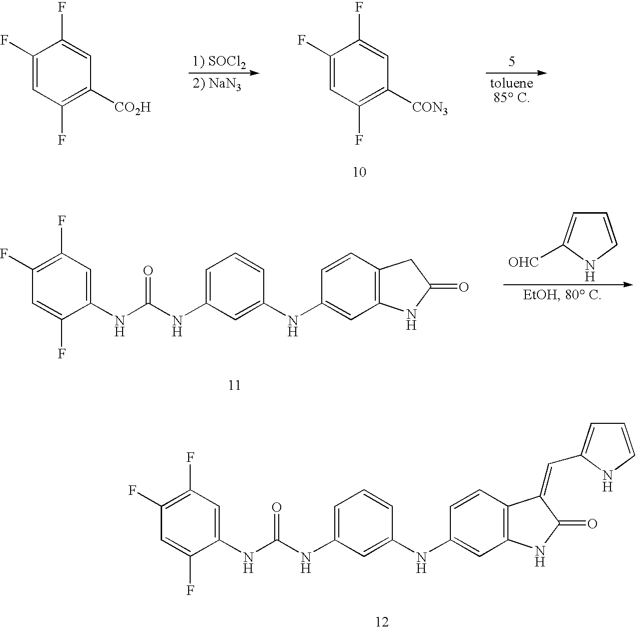

### Synthesis of 2,4,5-trifluorobenzoyl azide (10)

To a solution of 2,4,5-trifluorobenzoic acid (10.0 g, 0.057 mol) in toluene (150 mL) is added SOCl₂ (12.5 mL, 0.171 mol). The mixture is heated at reflux overnight. All the solvent is removed and the crude is dried in vacuo for an hour to remove residual SOCl₂. The crude is then dissolved in acetone (100 ml) and cooled to 0° C. A solution of NaN₃ (4.5 g, 0.0684 mol) in water (20 mL) is added slowly. The resulting mixture is warmed to room temperature and stirred for 2 hr. Acetone is removed. The mixture is extracted with EtOAc (3×150 mL). The combined organic layer is dried over Na₂SO₄, filtered and concentrated. The crude is purified by column chromatography (EtOAc/Hexane, gradient, 0-20%) to give the desired product as oil. ¹H NMR (400 MHz, CDCl₃) δ 7.90-7.70 (m, 1H), 7.10-7.00 (m, 1H).

### Synthesis of 1-[3-(2-oxo-2,3-dihydro-1H-indol-6-ylamino)phenyl]-3-(2,4,5-trifluoro-phenyl)urea (11)

To a suspension of 6-(3-aminophenylamino)-1,3-dihydroindol-2-one (5, 310 mg, 1.30 mmol) in toluene (15 mL) is added 2,4,5-trifluorobenzoyl azide (392 mg, 1.95 mmol)). The mixture is heated at 80° C. overnight. After cooling to room temperature, the mixture is concentrated and the crude is purified by column chramatography (silica gel, EtOAc/hexane, gradient, 0-100%) to give the desired product. LC-MS (m/z) 413.1 (M⁺+1).

### Synthesis of 1-{3-[2-oxo-3-(1H-pyrrol-2-ylmethylene)-2,3-dihydro-1H-indol-6-ylamino]phenyl}-3-(2,4,5-trifluorophenyl)urea (12)

To a suspension of 1-[3-(2-oxo-2,3-dihydro-1H-indol-6-ylamino)phenyl]-3-(2,4,5-trifluorophenyl)urea (543 mg, 1.32 mmol) in ethanol (25 mL) is added pyrrole-2-carboxaldehyde (150 mg, 1.58 mmol) and piperidine (0.13 ml, 1.32 mmol). The reaction is heated at reflux for 2 hr. The mixture is concentrated and purified by column chromatography (silica gel, EtOAc/hexane, gradient, 0-100%). The product is suspended in MeOH and filtered. The solid is ished with minimal MeOH several times to give 540 mg of the desired product. M.pt. 224-226° C.; ¹H NMR (400 MHz, DMSO-d₆) δ 10.73 (s, 1H), 9.02 (s, 1H), 8.65 (s, 1H), 8.33 (s, 1H), 8.25-8.15 (m, 1H), 7.65-7.55 (m, 1H), 7.50-7.40 (m, 2H), 7.35-7.30 (m, 1H), 7.30-7.25 (m, 1H), 7.16 (t, 1H), 6.88 (dd, 1H), 6.80-6.70 (m, 3H), 6.70-6.60 (m, 1H), 6.35-6.25 (m, 1H); LC-MS (m/z) 490.2 (M⁺+1).

By repeating the procedures described in the above examples, using appropriate starting materials, the following compounds of Formula I, as identified in Table 1, are obtained.

TABLE 1

| Com- | | |
|---|---|---|
| pound | | Physical Data |
| Number | Structure | ¹H NMR and/or MS (m/z) |
| 4 | 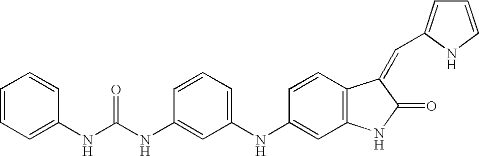 | LC-MS (m/z) [M⁺ + 1] 436.2 |
| 5 | 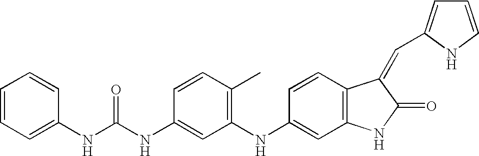 | LC-MS (m/z) [M⁺ + 1] 450.2 |
| 6 | 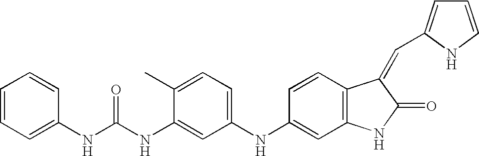 | LC-MS (m/z) [M⁺ + 1] 450.2 |
| 7 | 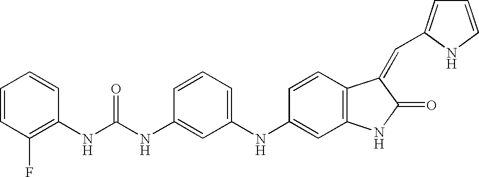 | ¹H NMR (400 MHz, DMSO-d₆) δ 13.16 (s, 1H), 10.77 (s, 1H), 9.03 (s, 1H), 8.50 (s, 1H), 8.36 (s, 1H), 8.15 (dt, 1H), 7.48 (s, 1H), 7.47 (d, 1H), 7.36 (t, 1H), 7.26 (s, 1H), 7.23 (ddd, 1H), 7.17 (t, 1H), 7.13 (dd, 1H), 6.98 (dq, 1H), 6.87 (dd, 1H), 6.73-6.71 (m, 3H), 6.66 (d, 1H), 6.30 (dt, 1H); LC-MS (m/z) [M⁺ + 1] 454.2 |
| 8 | 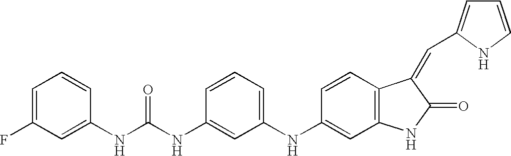 | LC-MS (m/z) [M⁺ + 1] 454.2 |
| 9 | 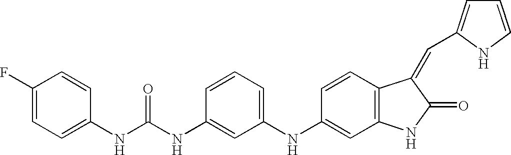 | LC-MS (m/z) [M⁺ + 1] 454.2 |
| 10 | 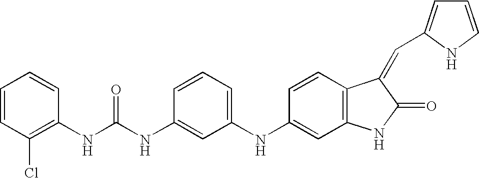 | LC-MS (m/z) [M⁺ + 1] 470.1 |
| 11 | 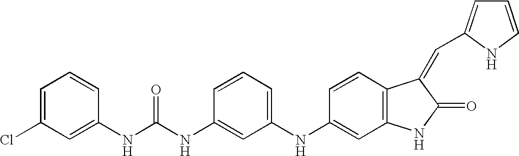 | LC-MS (m/z) [M⁺ + 1] 470.1 |
| 12 | 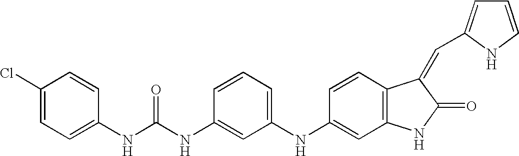 | LC-MS (m/z) [M⁺ + 1] 470.1 |
| 13 | 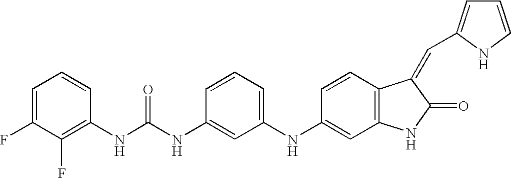 | LC-MS (m/z) [M⁺ + 1] 472.1 |
| 14 | 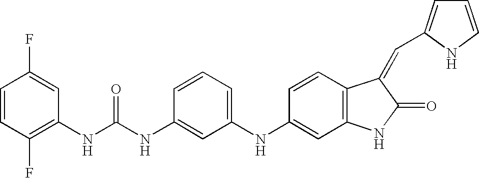 | ¹H (400 MHz, DMSO-d₆) δ 13.15 (s, 1H), 10.75 (s, 1H), 9.10 (s, 1H), 8.70 (s, 1H), 8.40 (s, 1H), 8.00 (s, 1H), 7.50 (d, 2H), 7.30 (m, 4H), 7.15 (m, 1H), 6.90 (m, 2H), 6.70 (m, 3H), 6.25 (s, 1H); LC-MS (m/z) [M⁺ + 1] 472.1 |
| 15 | 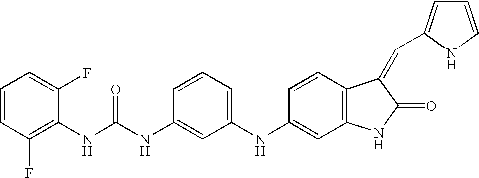 | LC-MS (m/z) [M⁺ + 1] 472.1 |
| 16 | 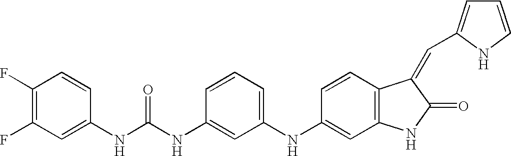 | ¹H NMR (400 MHz, DMSO-d₆) δ 13.15 (s, 1H), 10.79 (s, 1H), 9.02 (s, 1H), 8.86 (s, 1H), 8.32 (s, 1H), 7.70-7.68 (m, 1H), 7.66 (ddd, 1H), 7.47 (s, 1H), 7.46 (d, 1H), 7.36 (d, 1H), 7.34 (d, 1H), 7.25 (d, 1H), 7.14 (t, 1H), 7.14-7.12 (m, 1H), 6.88 (dd, 1H), 6.73-6.70 (m, 2H), 6.64 (d, 1H), 6.30 (dt, 1H); LC-MS (m/z) [M⁺ + 1] 472.1 |
| 17 | 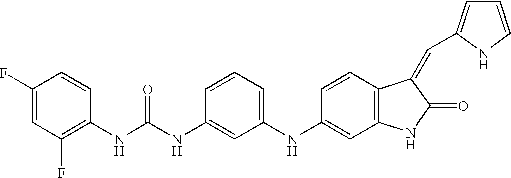 | ¹H NMR (400 MHz, DMSO-d₆) δ 13.15 (s, 1H), 10.77 (s, 1H), 8.99 (s, 1H), 8.48 (s, 1H), 8.36 (s, 1H), 8.07 (ddd, 1H), 7.48 (s, 1H), 7.46 (d, 1H), 7.35 (s, 1H), 7.34 (t, 1H), 7.32 (t, 1H), 7.26 (s, 1H), 7.14 (t, 1H), 7.02 (dt, 1H), 6.87 (dd, 1H), 6.72-6.70 (m, 2H), 6.65 (d, 1H), 6.30 (dt, 1H); LC-MS (m/z) [M⁺ + 1] 472.1 |
| 18 | 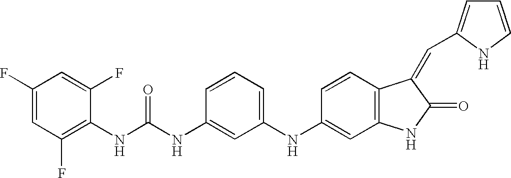 | LC-MS (m/z) [M⁺ + 1] 490.1 |
| 19 | 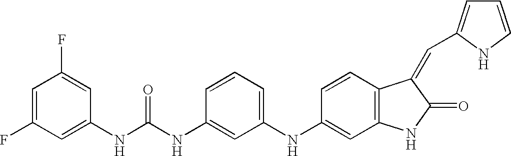 | LC-MS (m/z) [M⁺ + 1] 472.1 |
| 20 | 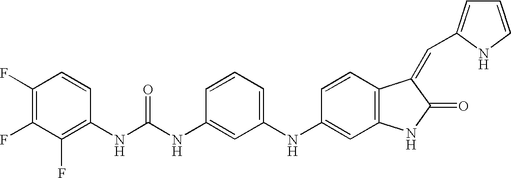 | ¹H NMR (400 MHz, DMSO-d₆) δ 13.10 (s, 1H), 10.74 (s, 1H), 9.01 (s, 1H), 8.62 (s, 1H), 8.33 (s, 1H), 7.88 (m, 1H), 7.47 (s, 1H), 7.46 (d, 1H), 7.34 (brs, 1H), 7.27-7.24 (m, 2H), 7.15 (t, 1H), 6.88 (dd, 1H), 6.73-6.71 (m, 3H), 6.65 (d, 1H), 6.30 (dt, 1H); LC-MS (m/z) [M⁺ + 1] 490.1 |
| 21 | 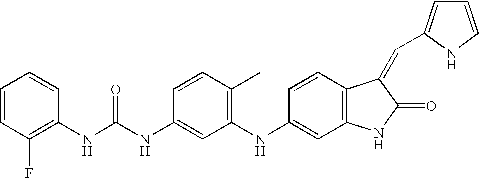 | ¹H NMR (DMSO-d₆) δ 13.10 (s, 1H), 10.69 (s, 1H), 8.96 (s, 1H), 8.43 (d, 1H), 8.13 (ddd, 1H), 7.57 (s, 1H), 7.44 (m, 2H), 7.41 (d, 1H), 7.22 (m, 2H), 7.11 (m, 2H), 7.00 (m, 2H), 6.70 (m, 1H), 6.59 (dd, 1H), 6.49 (d, 1H), 6.301 (m, 1H), 2.16 (s, 3H); LC-MS (m/z) [M⁺ + 1] 468.2 |
| 22 | 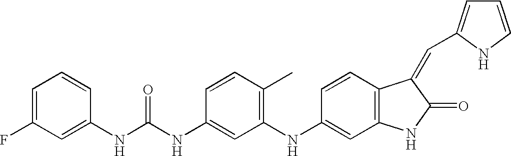 | LC-MS (m/z) [M⁺ + 1] 468.2 |
| 23 | 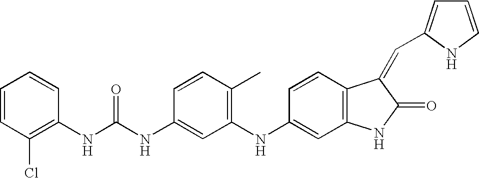 | LC-MS (m/z) [M⁺ + 1] 484.2 |
| 24 | 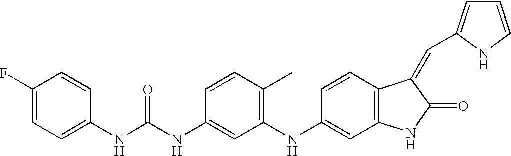 | LC-MS (m/z) [M⁺ + 1] 468.2 |
| 25 | 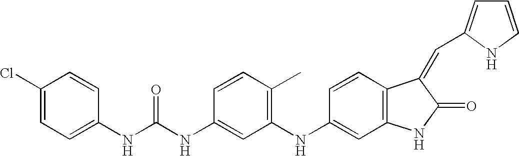 | LC-MS (m/z) [M⁺ + 1] 484.2 |
| 26 | 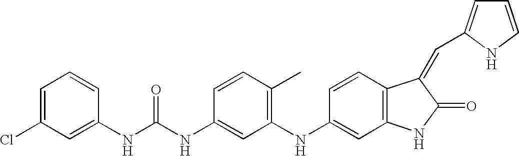 | ¹H NMR (DMSO-d₆) δ 13.17 (s, 1H), 10.69 (s, 1H0, 8.75 (s, 1H), 8.63 (s, 1H), 7.67 (t, 1H), 7.57 (s, 1H), 7.43 (m, 2H), 7.39 (d, 1H), 7.26 (m, 3H), 7.11 (d, 1H), 7.00 (m, 2H), 6.70 (m, 1H), 6.58 (dd, 1H), 6.47 (d, 1H), 6.30 (m, 1H), 2.15 (s, 3H); LC-MS (m/z) [M⁺ + 1] 484.2 |
| 27 | 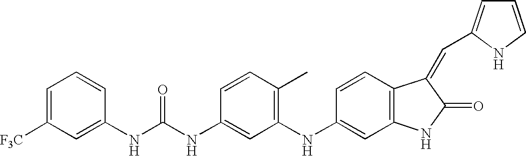 | LC-MS (m/z) [M⁺ + 1] 518.2 |
| 28 | 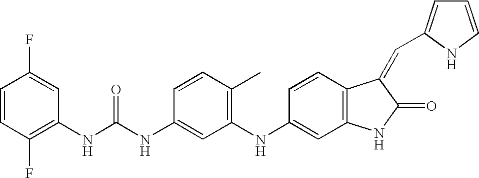 | LC-MS (m/z) [M⁺ + 1] 486.2 |
| 29 | 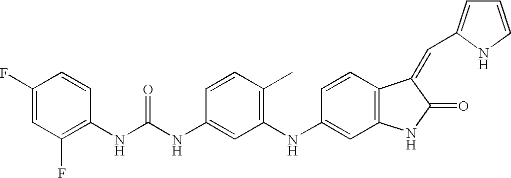 | LC-MS (m/z) [M⁺ + 1] 486.2 |
| 30 | 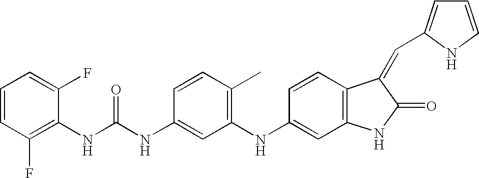 | LC-MS (m/z) [M⁺ + 1] 486.2 |
| 31 | 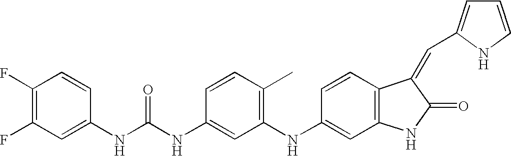 | LC-MS (m/z) [M⁺ + 1] 486.2 |
| 32 | 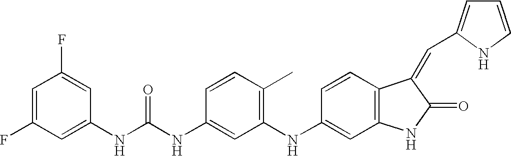 | LC-MS (m/z) [M⁺ + 1] 486.2 |
| 33 | 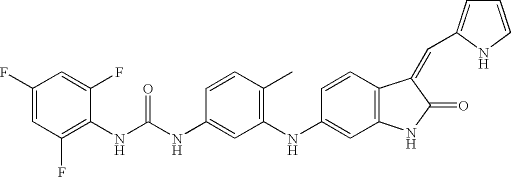 | LC-MS (m/z) [M⁺ + 1] 504.2 |
| 34 | 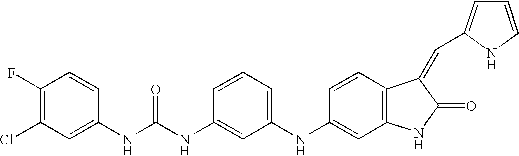 | ¹H NMR (400 MHz, DMSO-d₆) δ 13.15 (s, 1H), 10.77 (s, 1H), 8.82 (s, 1H), 8.72 (s, 1H), 8.35 (s, 1H), 7.78 (dd, 1H), 7.48 (s, 1H), 7.46 (d, 1H), 7.35 (s, 1H), 7.33 (dd, 1H), 7.30 (dt, 1H), 7.28 (t, 1H), 7.17 (t, 1H), 6.87 (dd, 1H), 6.72-6.70 (m, 3H), 6.64 (d, 1H), 6.30 (dt, 1H); LC-MS (m/z) [M⁺ + 1] 488.1 |
| 35 | 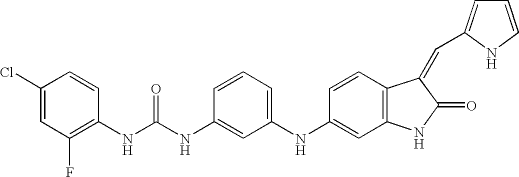 | LC-MS (m/z) [M⁺ + 1] 488.1 |
| 36 | 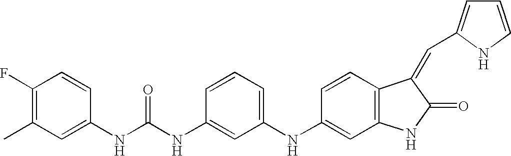 | ¹H NMR (400 MHz, DMSO-d₆) δ 13.16 (s, 1H), 10.76 (s, 1H), 8.61 (s, 1H), 8.55 (s, 1H), 8.33 (s, 1H), 7.47 (s, 1H), 7.46 (d, 1H), 7.35 (brs, 1H), 7.34 (dd, 1H), 7.26 (s, 1H), 7.23 (dd, 1H), 7.13 (t, 1H), 7.03 (t, 1H), 6.87 (d, 1H), 6.72-6.69 (m, 3H), 6.64 (d, 1H), 6.30 (dt, 1H), 2.20 (s, 3H); LC-MS (m/z) [M⁺ + 1] 468.2 |
| 37 | 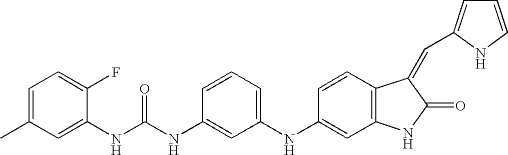 | ¹H NMR (400 MHz, DMSO-d₆) δ 10.72 (s, 1H), 8.99 (s, 1H), 8.41 (s, 1H), 8.30 (s, 1H), 7.97 (dd, 1H), 7.47 (s, 1H), 7.46 (d, 1H), 7.35 (s, 1H), 7.26 (s, 1H), 7.15 (t, 1H), 7.08 (t, 1H), 6.89 (d, 1H), 6.79-6.77 (m, 1H), 6.73-6.71 (m, 3H), 6.66 (d, 1H), 6.30 (dt, 1H), 2.23 (s, 3H); LC-MS (m/z) [M⁺ + 1] 468.2 |
| 38 | 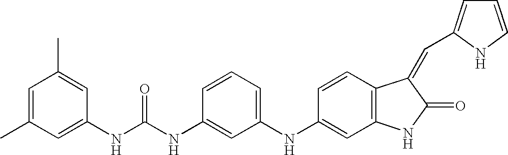 | ¹H NMR (400 MHz, DMSO-d₆) δ 13.15 (s, 1H), 10.73 (s, 1H), 8.56 (s, 1H), 8.43 (s, 1H), 8.30 (s, 1H), 7.47 (s, 1H), 7.46 (d, 1H), 7.35 (t, 1H), 7.26 (dd, 1H), 7.13 (t, 1H), 7.05 (d, 1H), 6.86 (dd, 1H), 6.73-6.69 (m, 3H), 6.65 (d, 1H), 6.60 (s, 1H), 6.30 (dt, 1H), 2.22 (s, 6H); LC-MS (m/z) [M⁺ + 1] 464.2 |
| 39 | 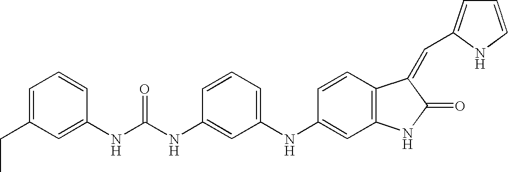 | LC-MS (m/z) [M⁺ + 1] 464.2 |
| 40 | 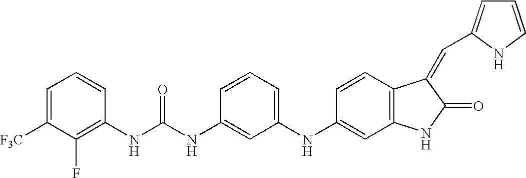 | ¹H (400 MHz, DMSO-d₆) δ 13.15 (s, 1H), 10.75 (s, 1H), 9.10 (s, 1H), 8.80 (s, 1H), 8.45 (s, 1H), 8.40 (s, 1H), 7.50 (s, 1H), 7.30 (m, 2H), 7.25 (s, 1H), 7.15 (m, 1H), 6.85 (m, 1H), 6.75 (m, 2H), 6.65 (s, 1H), 6.25 (s, 1H); LC-MS (m/z) [M⁺ + 1] 522.1 |
| 41 | 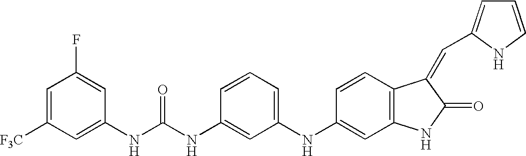 | ¹H NMR (400 MHz, DMSO-d₆) δ 13.15 (s, 1H), 10.77 (s, 1H), 9.19 (s, 1H), 8.88 (s, 1H), 8.37 (s, 1H), 7.69 (s, 1H), 7.60 (dt, 1H), 7.48 (s, 1H), 7.47 (d, 1H), 7.35 (t, 1H), 7.26 (t, 1H), 7.23 (dd, 1H), 7.16 (t, 1H), 6.89 (dd, 1H), 6.75 (dd, 1H), 6.73 (dd, 1H), 6.71 (d, 1H), 6.64 (d, 1H), 6.30 (dt, 1H); LC-MS (m/z) [M⁺ + 1] 522.1 |
| 42 | 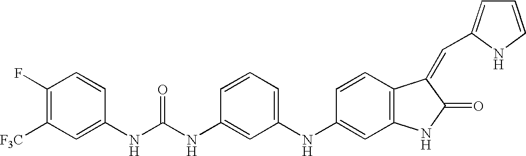 | ¹H NMR (400 MHz, DMSO-d₆) δ 13.16 (s, 1H), 10.76 (s, 1H), 8.99 (s, 1H), 8.78 (s, 1H), 8.36 (s, 1H), 8.00 (dd, 1H), 7.62 (dt, 1H), 7.48 (s, 1H), 7.46 (d, 1H), 7.41 (t, 1H), 7.35 (s, 1H), 7.26 (s, 1H), 7.15 (t, 1H), 6.89 (dd, 1H), 6.74-6.71 (m, 3H), 6.64 (d, 1H), 6.30 (dt, 1H); LC-MS (m/z) [M⁺ + 1] 522.1 |
| 43 | 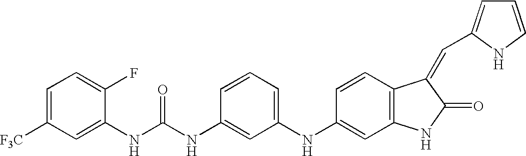 | ¹H (400 MHz, DMSO-d₆) δ 13.15 (s, 1H), 10.75 (s, 1H), 9.15 (s, 1H), 8.85 (s, 1H), 8.65 (d, 1H), 8.40 (s, 1H), 7.50 (m, 3H), 7.35 (m, 2H), 7.25 (s, 1H), 7.15 (m, 1H), 6.90 (m, 1H), 6.70 (m, 2H), 6.65 (s, 1H), 6.30 (s, 1H); LC-MS (m/z) [M⁺ + 1] 522.1 |
| 44 | 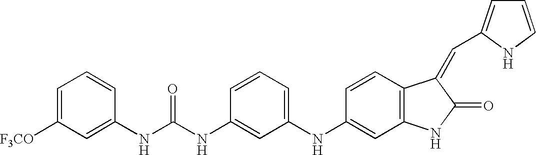 | LC-MS (m/z) [M⁺ + 1] 520.2 |
| 45 | 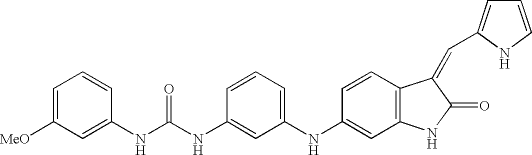 | LC-MS (m/z) [M⁺ + 1] 466.2 |
| 46 | 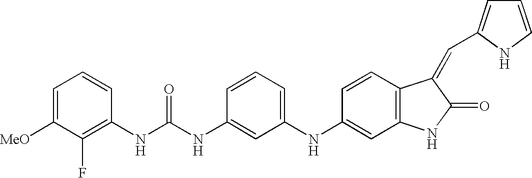 | LC-MS (m/z) [M⁺ + 1] 484.2 |
| 47 | 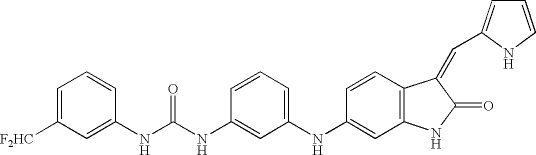 | LC-MS (m/z) [M⁺ + 1] 486.2 |
| 48 | 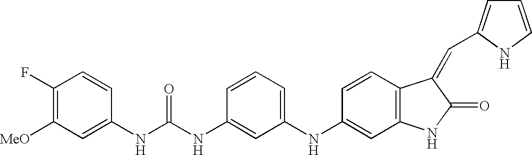 | LC-MS (m/z) [M⁺ + 1] 484.2 |
| 49 | 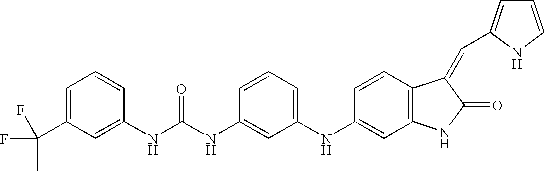 | ¹H (400 MHz, DMSO) δ 10.72 (s, 1 H), 8.82 (s, 1 H), 8.67 (s, 1 H), 8.31 (s, 1 H), 7.75 (s, 1 H), 7.50-7.45 (m, 3 H), 7.40-7.34 (m, 2 H), 7.28-7.24 (m, 1 H), 7.18-7.10 (m, 2 H), 6.89 (dd, 1 H), 6.75-6.70 (m, 3 H), 6.66 (d, 1 H), 6.32-6.28 (m, 1 H); LC-MS (m/z) [M⁺ + 1] 500.2 |
| 50 | 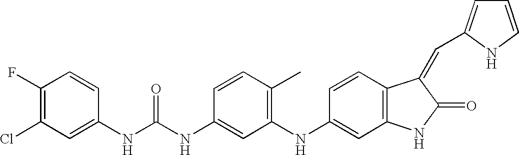 | ¹H (400 MHz, DMSO) δ 13.15 (s, 1H), 10.73 (s, 1H), 8.75 (s, 1H), 8.66 (s, 1H), 7.77 (d, J = 4.3 Hz, 1H), 7.59 (s, 1H), 7.42 (m, 3H), 7.29 (m, 3H), 7.09 (d, J = 8.4 Hz, 1H), 7.00 (m, 1H), 6.70 (s, 1H), 6.60 (m, 1H), 6.45 (s, 1H), 6.30 (s, 1H), 2.15 (s, 1H); LC-MS (m/z) [M⁺ + 1] 502.1 |
| 51 | 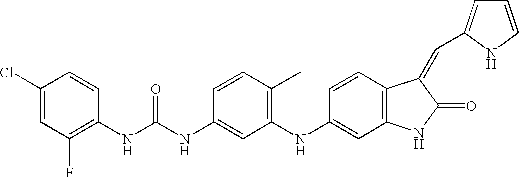 | LC-MS (m/z) [M⁺ + 1] 503.1 |
| 52 | 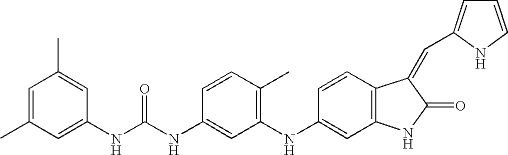 | ¹H (400 MHz, DMSO) δ 13.10 (s, 1H), 10.70 (s, 1H), 8.55 (s, 1H), 8.38 (s, 1H), 7.55 (s, 1H), 7.40 (m, 3H), 7.25 (s, 1H), 7.10 (d, J = 8.2 Hz, 1H), 7.00 (m, 3H), 6.70 (s, 1H), 6.55 (d, J = 8.2 Hz, 2H), 6.47 (s, 1H), 6.30 (s, 1H), 2.20 (s, 6H), 2.18 (s, 3H); LC-MS (m/z) [M⁺ + 1] 477 |
| 53 | 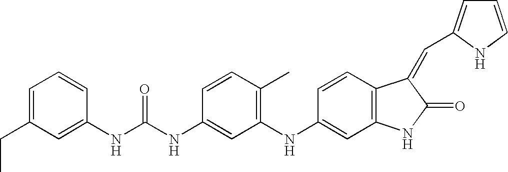 | LC-MS (m/z) [M⁺ + 1] 478.2 |
| 54 | 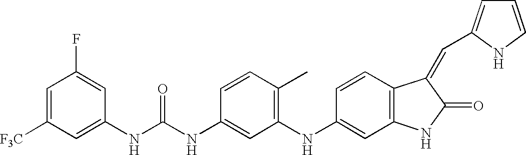 | ¹H (400 MHz, DMSO) δ 13.15 (s, 1H), 10.75 (s, 1H), 9.17 (s, 1H), 8.80 (s, 1H), 7.65 (m, 3H), 7.38 (m, 3H), 7.15 (d, J = 8.2 Hz, 1H), 7.05 (d, J = 8.2 Hz, 1H), 6.70 (s, 1H), 6.58 (d, J = 8.2 Hz, 1H), 6.47 (s, 1H), 6.30 (s, 1H), 2.15 (s, 3H); LC-MS (m/z) [M⁺ + 1] 536.1 |
| 55 | 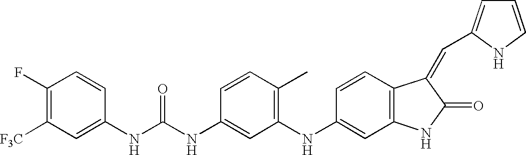 | ¹H (400 MHz, DMSO) δ 13.15 (s, 1H), 10.75 (s, 1H), 8.95 (s, 1H), 8.70 (s, 1H), 7.95 (m, 1H), 7.60 (m, 2H), 7.45 (m, 4H), 7.25 (s, 1H), 7.10 (d, J = 8.2 Hz, 1H), 7.05 (d, J = 8.2 Hz, 1H), 6.70 (s, 1H), 6.56 (d, J = 8.1 Hz, 1H), 6.42 (s, 1H), 6.25 (s, 1H), 2.15 (s, 3H); LC-MS (m/z) [M⁺ + 1] 536.1 |
| 56 | 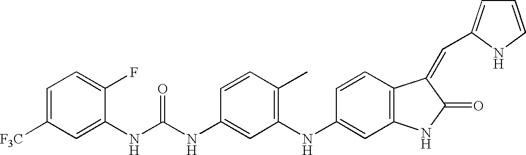 | ¹H (400 MHz, DMSO-d₆) δ 13.15 (s, 1H), 12.10 (br s, 1H), 10.70 (s, 1H), 9.45 (s, 1H), 9.10 (s, 1H), 8.80 (s, 1H), 8.60 (d, 1H), 7.60 (s, 1H), 7.50 (m, 3H), 7.40 (s, 1H), 7.25 (d, 1H), 7.15 (d, 1H), 7.05 (m, 1H), 6.70 (s, 1H), 6.60 (d, 1H), 6.50 (s, 1H), 6.30 (m, 1H), 2.10 (s, 3H); LC- MS (m/z) [M⁺ + 1] 536.1 |
| 57 | 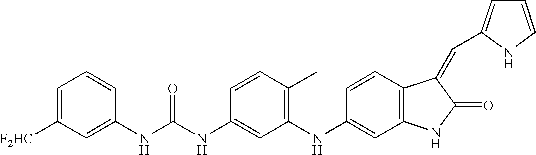 | LC-MS (m/z) [M⁺ + 1] 500.2 |
| 58 | 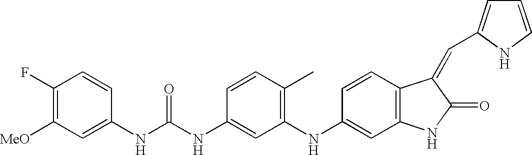 | LC-MS (m/z) [M⁺ + 1] 498.2 |
| 59 | 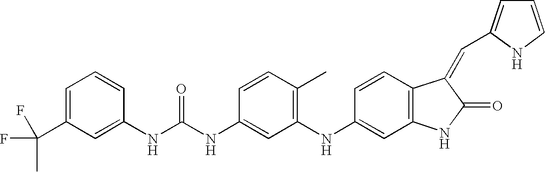 | LC-MS (m/z) [M⁺ + 1] 514.2 |
| 60 | 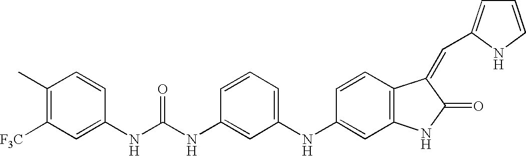 | ¹H NMR(400 MHz, DMSO-d₆) δ 13.15 (s, 1H), 10.76 (s, 1H), 8.86 (s, 1H), 8.70 (s, 1H), 8.36 (s, 1H), 7.92 (d, 1H), 7.48 (s, 2H), 7.45 (d, 1H), 7.35 (s, 1H), 7.32 (d, 1H), 7.26 (brs, 1H), 7.14 (t, 1H), 6.87 (dd, 1H), 6.74-6.70 (m, 3H), 6.64 (d, 1H), 6.30 (dt, 1H), 2.36 (s, 3H); LC-MS (m/z) [M⁺ + 1] 518.2 |
| 61 | 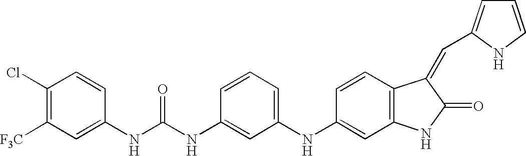 | ¹H NMR(400 MHz, DMSO-d₆) δ 13.15 (s, 1H), 10.76 (s, 1H), 9.12 (s, 1H), 8.82 (s, 1H), 8.37 (s, 1H), 8.10 (s, 1H), 7.61 (s, 2H), 7.48 (s, 1H), 7.46 (d, 1H), 7.35 (t, 1H), 7.26 (t, 1H), 7.15 (t, 1H), 6.88 (dd, 1H), 6.74 (dd, 1H), 6.72 (d, 1H), 6.70 (d, 1H), 6.64 (d, 1H), 6.30 (dt, 1H); LC-MS (m/z) [M⁺ + 1] 538.1 |
| 62 | 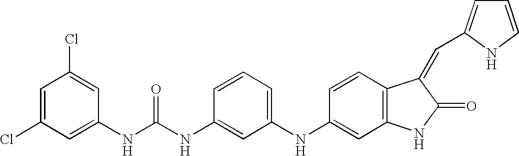 | ¹H NMR(400 MHz, DMSO-d₆) δ 13.16 (s, 1H), 10.77 (s, 1H), 9.00 (s, 1H), 8.85 (s, 1H), 8.36 (s, 1H), 7.52 (d, 2H), 7.48 (s, 1H), 7.46 (d, 1H), 7.34 (t, 1H), 7.26 (brs, 1H), 7.16 (t, 1H), 7.15 (t, 1H), 6.88 (dd, 1H), 6.74-6.71 (m, 3H), 6.64 (d, 1H), 6.30 (dt, 1H); LC-MS (m/z) [M⁺ + 1] 504.1 |
| 63 | 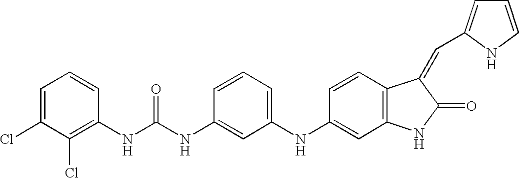 | LC-MS (m/z) [M⁺ + 1] 504.1 |
| 64 | 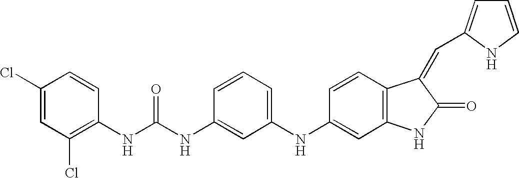 | ¹H NMR(400 MHz, DMSO-d₆) δ 13.16 (s, 1H), 10.77 (s, 1H), 9.40 (s, 1H), 8.36 (s, 1H), 8.19 (d, 1H), 7.63 (d, 1H), 7.48 (s, 1H), 7.47 (d, 1H), 7.38 (dd, 1H), 7.35 (t, 1H), 7.27 (s, 1H), 7.16 (t, 1H), 6.89 (dd, 1H), 6.73-6.71 (m, 3H), 6.66 (d, 1H), 6.30 (dt, 1H); LC-MS (m/z) [M⁺ + 1] 504.1 |
| 65 | 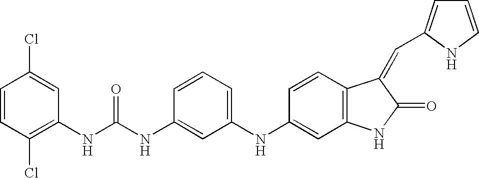 | ¹H NMR(400 MHz, DMSO-d₆) δ 13.16 (s, 1H), 10.76 (s, 1H), 9.48 (s, 1H), 8.43 (s, 1H), 8.36 (s, 1H), 8.32 (d, 1H), 7.50 (d, 1H), 7.49 (s, 1H), 7.48 (d, 1H), 7.34 (t, 1H), 7.26 (brs, 1H), 7.17 (t, 1H), 7.08 (dd, 1H), 6.91 (dd, 1H), 6.75-6.66 (m, 3H), 6.65 (d, 1H), 6.30 (dt, 1H); LC-MS (m/z) [M⁺ + 1] 504.1 |
| 66 | 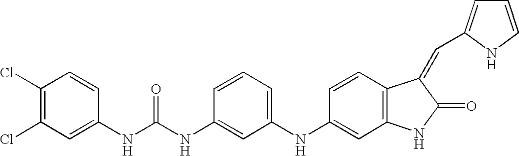 | ¹H NMR(400 MHz, DMSO-d₆) δ 13.15 (s, 1H), 10.74 (s, 1H), 8.91 (s, 1H), 8.75 (s, 1H), 8.32 (s, 1H), 7.87 (d, 1H), 7.49 (d, 1H), 7.47 (s, 1H), 7.46 (d, 1H), 7.34 (t, 1H), 7.30 (dd, 1H), 7.26 (s, 1H), 7.17 (t, 1H), 6.87 (dd, 1H), 6.73-6.71 (m, 3H), 6.64 (d, 1H), 6.30 (dt, 1H); LC-MS (m/z) [M⁺ + 1] 504.1 |
| 67 | 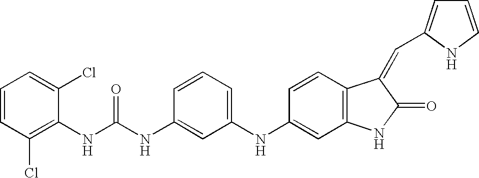 | ¹H NMR(400 MHz, DMSO-d₆) δ 13.15 (s, 1H), 10.75 (s, 1H), 8.92 (s, 1H), 8.32 (s, 1H), 8.14 (s, 1H), 7.54 (s, 1H), 7.52 (t, 1H), 7.46 (s, 1H), 7.45 (d, 1H), 7.35 (brs, 1H), 7.28 (t, 1H), 7.25 (brs, 1H), 7.17 (t, 1H), 6.89 (dd, 1H), 6.71-6.69 (m, 3H), 6.64 (d, 1H), 6.30 (dt, 1H); LC-MS (m/z) [M⁺ + 1] 504.1 |
| 68 | 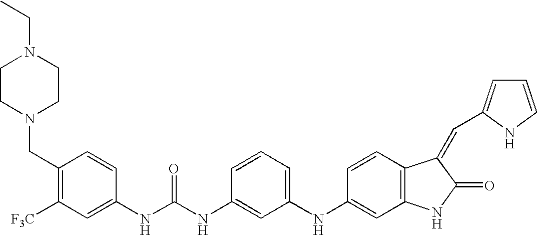 | ¹H (400 MHz, DMSO) δ 10.73 (s, 1H), 9.77 (br s, 1 H), 9.28 (s, 1 H), 8.02 (s, 1 H), 7.85 (br s, 1 H), 7.70-7.60 (m, 1 H), 7.55-7.45 (m, 2 H), 7.40-7.35 (m, 1 H), 7.30-7.25 (m, 1 H), 7.15 (t, 1 H), 6.95-6.85 (m, 1 H), 6.80-6.60 (m, 4 H), 6.35-6.28 (m, 1 H); LC-MS (m/z) [M⁺ + 1] 630.3 |
| 69 | 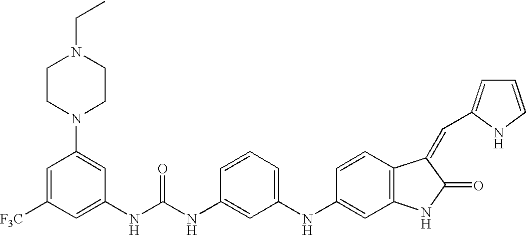 | ¹H (400 MHz, DMSO) δ 13.10 (s, 1H), 10.75 (s, 1H), 9.45 (s, 1H), 8.90 (s, 1H), 8.80 (s, 1H), 7.55 (m, 4H), 7.30 (s, 1H), 7.20 (d, 1H), 7.00 (m, 1H), 6.90 (s, 1H), 6.75 (s, 1H), 6.50 (s, 1H), 6.30 (s, 1H), 6.20 (s, 1H), 5.70 (s, 1H), 3.85 (m, 1H), 3.62 (m, 1H), 3.60-3.10 (m, 6H), 2.85 (q, 2H), 1.10 (t, 3H); LC-MS (m/z) [M⁺ + 1] 616 |
| 70 | 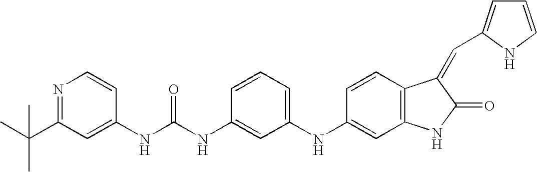 | LC-MS (m/z) [M⁺ + 1] 493.2 |
| 71 | 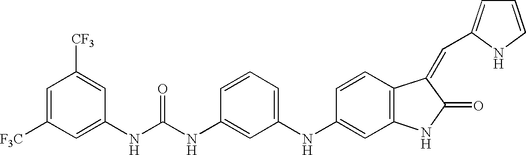 | ¹H NMR (400 MHz, DMSO-d₆) δ 13.15 (s, 1H), 10.77 (s, 1H), 9.50 (s, 1H), 9.08 (brs, 1H), 8.37 (s, 1H), 8.13 (s, 2H), 7.64 (s, 1H), 7.48 (s, 1H), 7.46 (d, 1H), 7.37 (brs, 1H), 7.26 (brs, 1H), 7.16 (t, 1H), 6.92 (dd, 1H), 6.76 (dd, 1H), 6.73 (dd, 1H), 6.70 (d, 1H), 6.65 (d, 1H), 6.29 (dt, 1H); LC-MS (m/z) [M⁺ + 1] 572.2 |
| 72 | 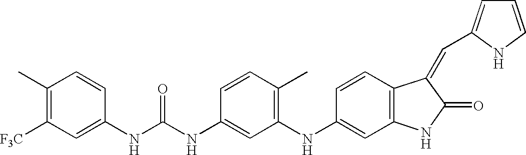 | ¹H (400 MHz, DMSO) δ 13.15 (s, 1H), 10.70 (s, 1H), 9.70 (s, 1H), 9.50 (s, 1H), 7.95 (s, 1H), 7.65 (s, 1H), 7.55 (m, 1H), 7.45 (m, 3H), 7.25 (m, 3H), 7.08 (m, 2H), 6.68 (s, 1H), 6.55 (d, J = 8.2 Hz, 1H), 6.40 (s, 1H), 6.25 (s, 1H), 2.35 (s, 3H), 2.15 (s, 3H); LC-MS (m/z) [M⁺ + 1] 532 |
| 73 | 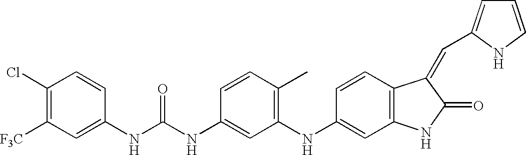 | ¹H (400 MHz, DMSO) δ 13.15 (s, 1H), 10.70 (s, 1H), 10.12 (s, 1H), 9.75 (s, 1H), 8.10 (s, 1H), 7.70 (m, 4H), 7.45 (m, 3H), 7.20 (m, 1H), 7.10 (m, 1H), 6.68 (s, 1H), 6.50 (d, J = 8.2 Hz, 1H), 6.40 (s, 1H), 6.28 (s, 1H), 2.18 (s, 3H); LC-MS (m/z) [M⁺ + 1] 552 |
| 74 | 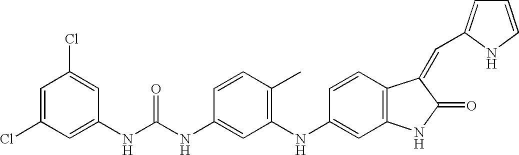 | LC-MS (m/z) [M⁺ + 1] 518.1 |
| 75 | 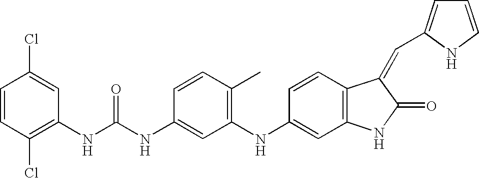 | LC-MS (m/z) [M⁺ + 1] 518.1 |
| 76 | 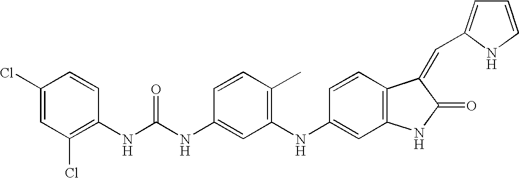 | LC-MS (m/z) [M⁺ + 1] 518.1 |
| 77 | 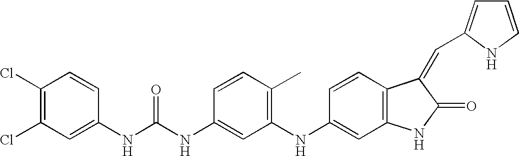 | ¹H (400 MHz, DMSO) δ 10.69 (s, 1H), 8.85 (s, 1 H), 8.68 (s, 1 H), 7.83 (d, 1 H), 7.56 (s, 1 H), 7.48 (d, 1 H), 7.44 (s, 1 H), 7.42 (d, 1 H), 7.39 (d, 1 H), 7.30 (dd, 1 H), 7.26-7.23 (m, 1 H), 7.10 (d, 1 H), 7.00 (dd, 1 H), 6.69 (qt, 1 H), 6.57 (dd, 1 H), 6.46 (d, 1 H), 6.32-6.28 (m, 1 H), 2.15 (s, 3 H); LC-MS (m/z) [M⁺ + 1] 518.1 |
| 78 | 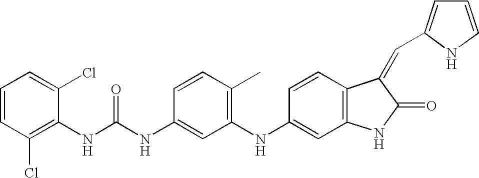 | LC-MS (m/z) [M⁺ + 1] 518.1 |
| 79 | 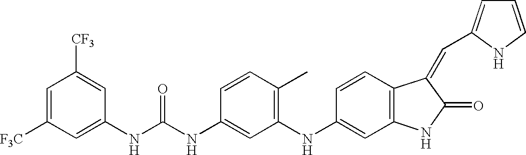 | ¹H (400 MHz, DMSO) δ 13.15 (s, 1H), 10.70 (s, 1H), 9.40 (s, 1H), 8.95 (s, 1H), 8.10 (s, 1H), 7.60 (s, 1H), 7.45 (m, 2H), 7.25 (s, 1H), 7.10 (m, 2H), 6.68 (s, 1H), 6.58 (m, 2H), 6.45 (s, 1H), 6.28 (s, 1H), 2.18 (s, 3H); LC-MS (m/z) [M⁺ + 1] 586 |
| 80 | 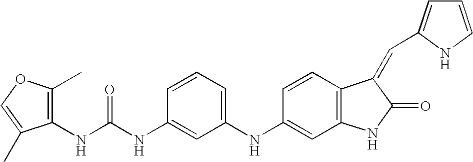 | LC-MS (m/z) [M⁺ + 1] 454.2 |
| 81 | 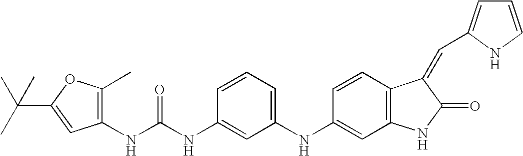 | LC-MS (m/z) [M⁺ + 1] 496.2 |
| 82 | 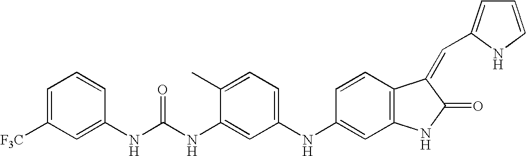 | ¹H NMR (400 MHz, DMSO-d₆) δ 13.17 (s, 1H), 10.7 (s, 1H), 9.41 (s, 1H), 8.24 (s, 1H), 8.00 (d, 1H), 7.73 (d, 1H), 7.54 (m, 2H), 7.44 (m, 2H), 7.31 (d, 1H), 7.25 (m, 1H), 7.08 (d, 1H), 6.77 (dd, 1H), 6.69 (m, 2H), 6.62 (d, 1H), 6.32 (m, 1H), 2.20 (s, 3H); LC-MS (m/z) [M⁺ + 1] 518.2 |
| 83 | 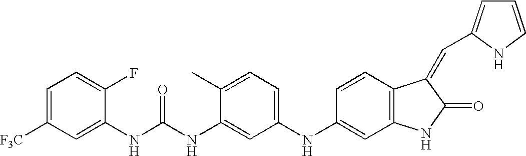 | LC-MS (m/z) [M⁺ + 1] 536.2 |
| 84 | 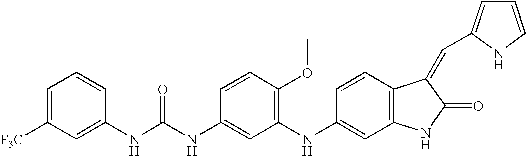 | ¹H NMR (400 MHz, DMSO-d₆) δ 13.17 (s, 1H), 10.72 (s, 1H), 8.88 (s, 1H), 8.58 (s, 1H), 7.96 (s, 1H), 7.57 (m, 2 H), 7.50 (d, 1H, 7.47 (s, 1H), 7.45 (d, 1H), 7.41 (d, 1H), 7.28 (m, 2H), 7.01 (dd, 1H), 6.95 (d, 1H), 6.73 (m, 2H), 6.64 (d, 1H), 6.31 (m, 1H), 3.80 (s, 3H); LC-MS (m/z) [M⁺ + 1] 534.2 |
| 85 | 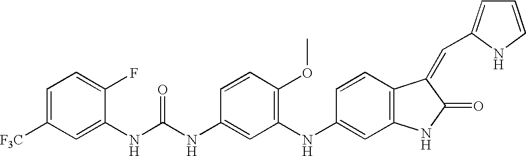 | ¹H NMR (DMSO-d₆) δ 13.18 (s, 1H), 10.73 (s, 1H), 8.99 (s, 1 H), 8.73 (d, 1H, 8.59 (dd, 1H), 7.58 (s, 1H), 7.47 (m, 3H), 7.36 (m, 2H), 7.26 (dd, 1H), 7.03 (dd, 1H), 6.96 (d, 1H), 6.73 (m, 2H), 6.64 (d, 1H), 6.31 (m, 1H), 3.80 (s, 3H); LC-MS (m/z) [M⁺ + 1] 552.2 |
| 86 | 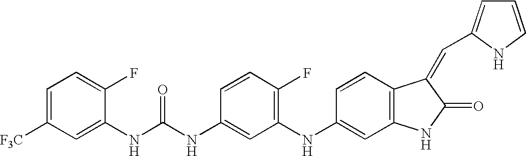 | ¹H (DMSO-d₆) δ 13.18 (s, 1H), 10.78 (s. 1H), 9.17 (s, 1H), 8.81 (s, 1H), 8.57 (d, 1H), 8.13 (s, 1H), 7.50 (m, 4H), 7.39 (m, 1H), 7.28 (s, 1H), 7.14 (t, 1H), 6.99 (m, 1H), 6.69 (m, 2H), 6.58 (s, 1H), 6.31 (m, 1H); LC-MS (m/z) [M⁺ + 1] 540.2. |
| 87 | 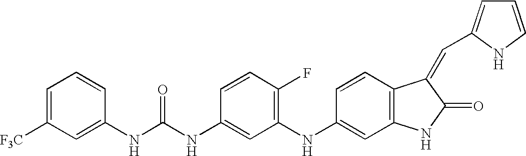 | ¹H NMR (DMSO-d₆) δ 13.17 (s, 1H), 10.77 (s, 1H), 8.97 (s, 1H), 8.80 (s, 1H), 8.11 (s, 1H), 7.96 (s, 1H), 7.51 (m, 5H), 7.29 (m, 2H), 7.15 (m, 1H), 7.00 (m, 1H), 6.72 (m, 2H), 6.58 (s, 1H), 6.32 (m, 1H); LC-MS (m/z) [M⁺ + 1] 522.2. |
| 88 | 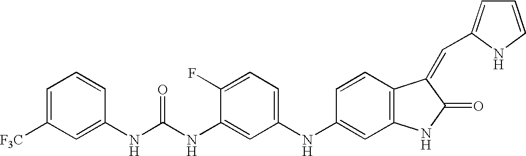 | ¹H NMR (DMSO-d₆) δ 13.17 (s, 1H), 10.72 (s, 1H), 9.43 (s, 1H), 8.60 (d, 1H), 8.30 (s, 1H), 8.01 (m, 2H), 7.54 (m, 2H), 7.46 (m, 2H), 7.33 (m, 1H), 7.26 (m, 1H), 7.15 (dd, 1H), 6.71 (m, 3H), 6.61 (d, 1H), 6.30 (m, 1H); LC-MS (m/z) [M⁺ + 1] 522.2 |
| 89 | 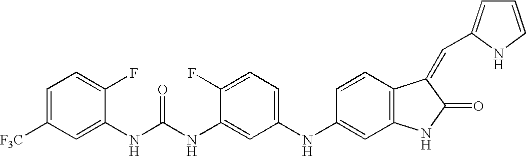 | ¹H NMR (DMSO-d₆) δ 13.17 (s, 1H), 10.77 (s, 1H), 9.45 (d, 1H), 9.20 (d, 1H), 8.37 (s, 1H), 8.10 (dd, 1H), 7.54 (m, 3H), 7.46 (m, 1H), 7.31 (m, 1H), 7.22 (dd, 1H), 6.83 (m, 1H), 6.77 (m, 1H), 6.73 (dd, 1H), 6.66 (d, 1H), 6.36 (m, 1H); LC-MS (m/z) [M⁺ + 1] 540.1 |
| 90 | 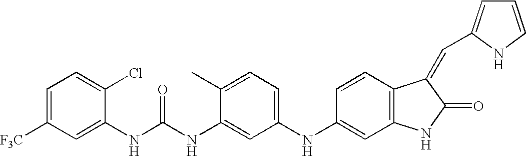 | ¹H NMR (DMSO-d₆) δ 13.17 (s, 1H), 10.75 (s, 1H), 9.06 (s, 1H), 8.80 (s, 1H), 8.67 (d, 1H), 8.31 (s, 1H), 7.78 (d, 1H), 7.72 (d, 1H), 7.49 (m, 2H), 7.42 (dd, 1H), 7.30 (dd, 1H), 7.15 (d, 1H), 6.70 (dd, 1H), 6.75 (m, 2H), 6.67 (d, 1H), 6.65 (m, 1H), 2.28 (s, 3H); LC-MS (m/z) [M⁺ + 1] 551.2 |
| 91 | 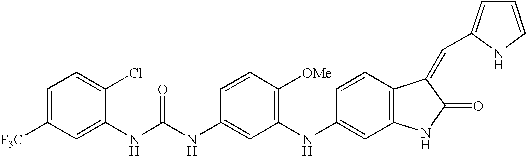 | LC-MS (m/z) [M⁺ + 1] 558.1 |
| 92 | 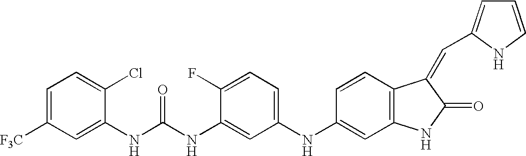 | LC-MS (m/z) [M⁺ + 1] 556.1 |
| 93 | 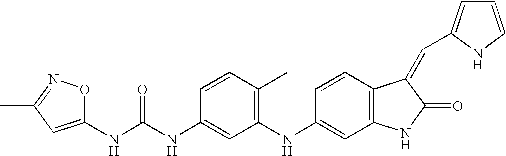 | ¹H (400 MHz, DMSO) δ 13.15 (s, 1H), 10.70 (s, 1H), 9.90 (s, 1H), 8.85 (s, 1H), 7.60 (s, 1H), 7.45 (d, 2H), 7.25 (s, 1H), 7.15 (d, 1H), 7.00 (d, 1H), 6.70 (s, 1H), 6.60 (d, 1H), 6.50 (s, 1H), 6.30 (s, 1H), 5.90 (s, 1H), 2.10 (s, 6H); LC-MS (m/z) [M⁺ + 1] 455 |
| 94 | 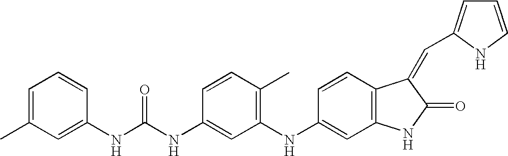 | LC-MS (m/z) [M⁺ + 1] 464.2 |
| 95 | 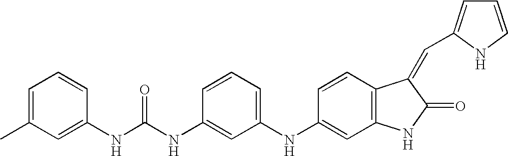 | LC-MS (m/z) [M⁺ + 1] 450.2 |
| 96 | 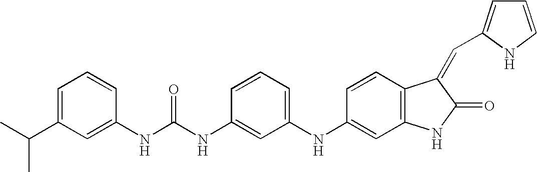 | LC-MS (m/z) [M⁺ + 1] 478.2 |
| 97 | 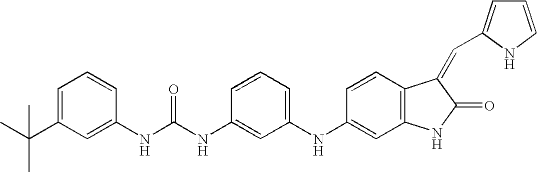 | LC-MS (m/z) [M⁺ + 1] 492.2 |
| 98 | 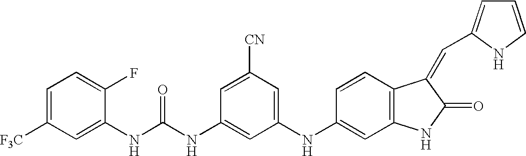 | LC-MS (m/z) [M⁺ + 1] 547.1 |
| 99 | 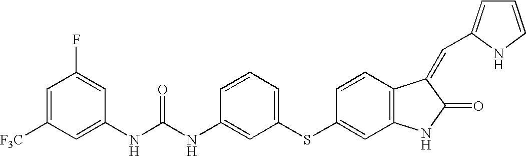 | LC-MS (m/z) [M⁺ + 1] 539.1 |
| 100 | 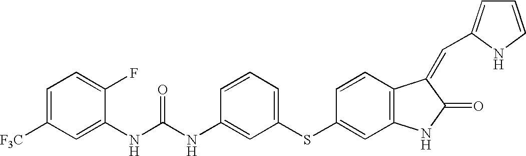 | LC-MS (m/z) [M⁺ + 1] 539.1 |
| 101 | 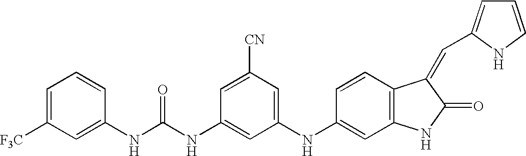 | LC-MS (m/z) [M⁺ + 1] 529.1 |
| 102 | 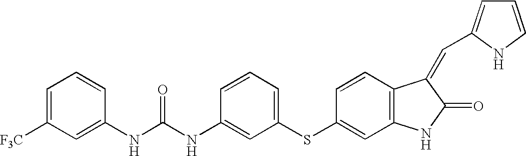 | LC-MS (m/z) [M⁺ + 1] 521.1 |
| 103 | 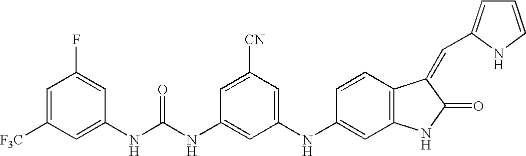 | LC-MS (m/z) [M⁺ + 1] 546.1 |
| 104 | 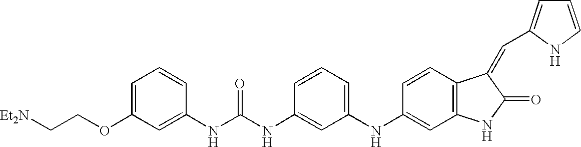 | ¹H (400 MHz, DMSO) δ 13.10 (s, 1H), 10.70 (s, 1H), 9.25 (s, 1H), 8.90 (s, 1H), 8.80 (s, 1H), 8.30 (s, 1H), 7.50 (m, 2H), 7.40 (m, 2H), 7.20 (m, 2H), 7.10 (m, 2H), 6.90 (m, 2H), 6.70 (m, 2H), 6.60 (m, 2H), 4.25 (m, 2H), 3.50 (m, 2H), 3.20 (m, 4H), 1.20 (t, 6H); LC-MS (m/z) [M⁺ + 1] 551 |
| 105 | 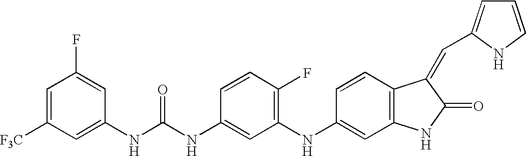 | LC-MS (m/z) [M⁺ + 1] 540.2 |
| 106 | 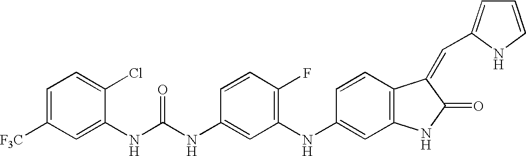 | LC-MS (m/z) [M⁺ + 1] 556.1 |
| 107 | 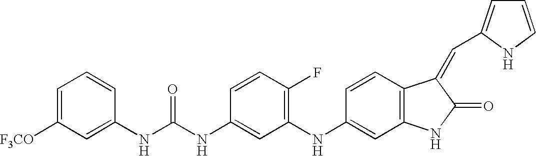 | ¹H NMR (400 MHz, DMSO-d₆) δ 10.73 (s, 1H), 8.89 (s, 1H), 8.73 (s, 1H), 8.07 (s, 1H), 7.63 (s, 1H), 7.49 (dd, 1H), 7.48 (s, 1H), 7.46 (d, 1H), 7.37 (t, 1H), 7.29-7.26 (m, 3H), 7.12 (dd, 1H), 6.97 (ddd, 1H), 6.93-6.91 (m, 2H), 6.72-6.70 (m, 1H), 6.57 (d, 1H), 6.30 (dt, 1H); LC-MS (m/z) [M⁺ + 1] 538.2 |
| 108 | 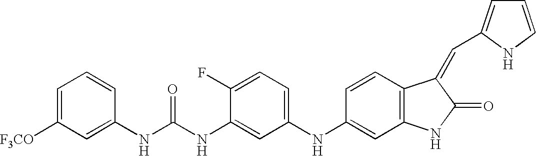 | LC-MS (m/z) [M⁺ + 1] 538.1 |
| 109 | 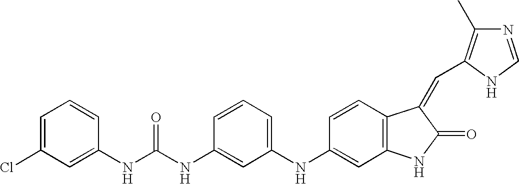 | LC-MS (m/z) [M⁺ + 1] 485.1 |
| 110 | 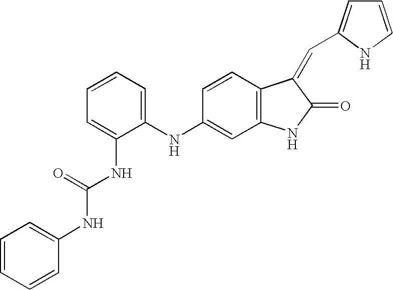 | LC-MS (m/z) [M⁺ + 1] 436.1 |
| 111 | 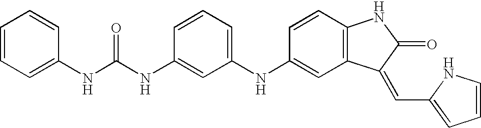 | LC-MS (m/z) [M⁺ + 1] 436.1 |
| 112 | 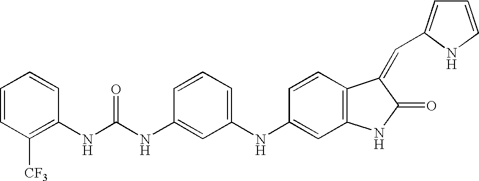 | LC-MS (m/z) [M⁺ + 1] 504.1 |
| 113 | 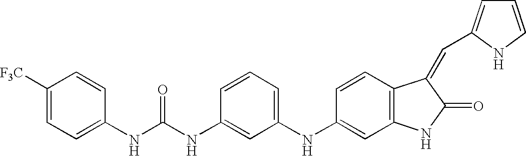 | LC-MS (m/z) [M⁺ + 1] 504.1 |
| 114 | 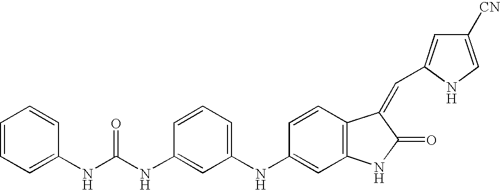 | LC-MS (m/z) [M⁺ + 1] 461.1 |
| 115 | 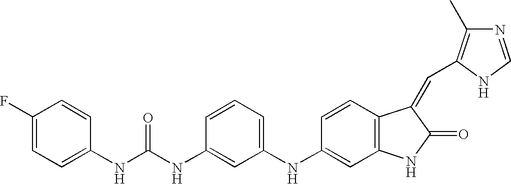 | LC-MS (m/z) [M⁺ + 1] 469.1 |
| 116 | 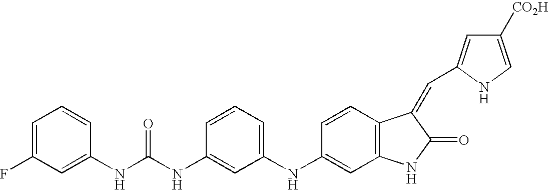 | LC-MS (m/z) [M⁺ + 1] 498.1 |
| 117 | 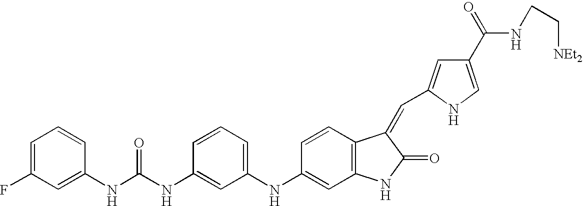 | LC-MS (m/z) [M⁺ + 1] 596.3 |
| 118 | 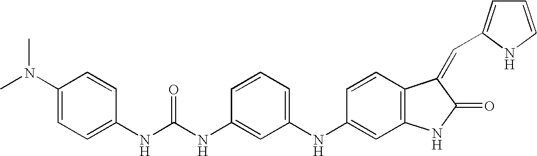 | LC-MS (m/z) [M⁺ + 1] 479.2 |
| 119 | 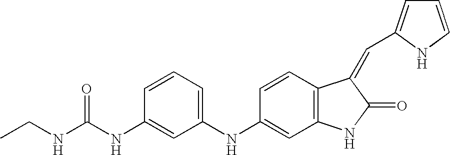 | LC-MS (m/z) [M⁺ + 1] 388.1 |
| 120 | 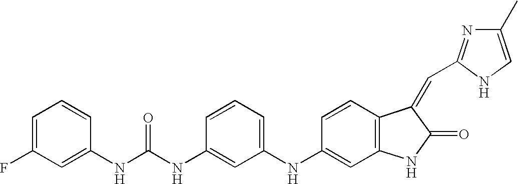 | LC-MS (m/z) [M⁺ + 1] 469.1 |
| 121 | 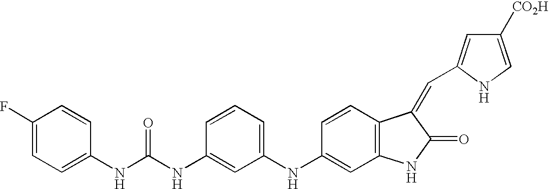 | LC-MS (m/z) [M⁺ + 1] 498.1 |
| 122 | 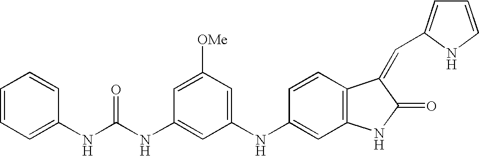 | LC-MS (m/z) [M⁺ + 1] 466.2 |
| 123 | 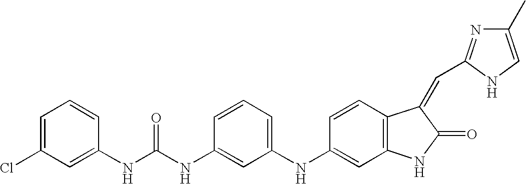 | LC-MS (m/z) [M⁺ + 1] 485.1 |
| 124 | 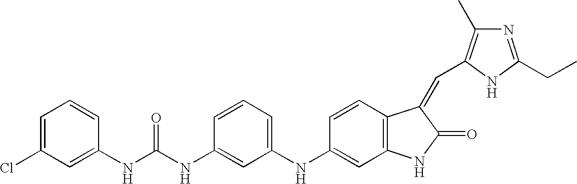 | LC-MS (m/z) [M⁺ + 1] 513.2 |
| 125 | 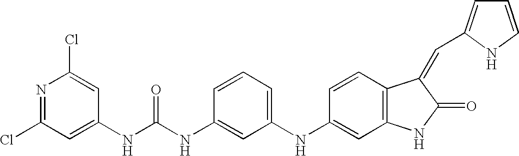 | LC-MS (m/z) [M⁺ + 1] 505.1 |
| 126 | 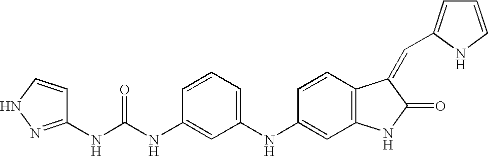 | LC-MS (m/z) [M⁺ + 1] 426.1 |
| 127 | 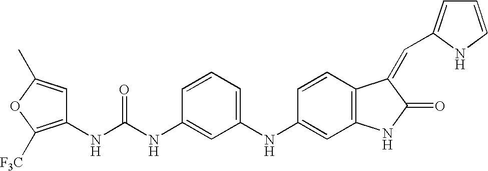 | LC-MS (m/z) [M⁺ + 1] 508.1 |
| 128 | 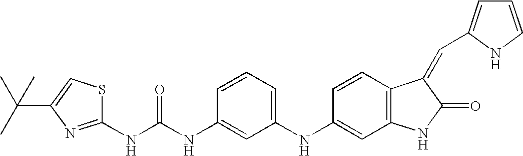 | LC-MS (m/z) [M⁺ + 1] 499.2 |
| 129 | 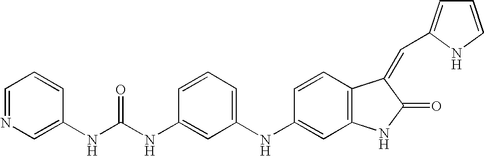 | LC-MS (m/z) [M⁺ + 1] 437.1 |
| 130 | 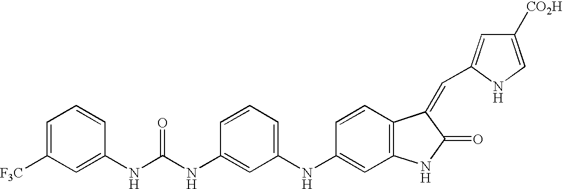 | LC-MS (m/z) [M⁺ + 1] 548.1 |
| 131 | 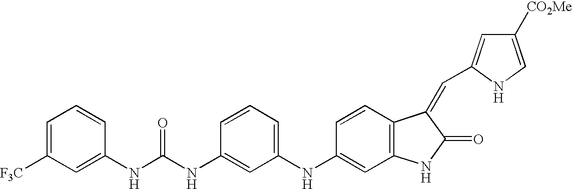 | LC-MS (m/z) [M⁺ + 1] 562.1 |
| 132 | 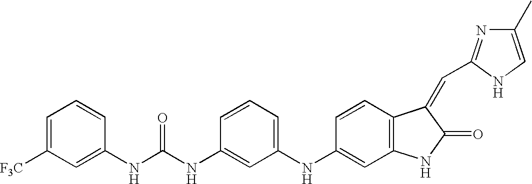 | LC-MS (m/z) [M⁺ + 1] 519.1 |
| 133 | 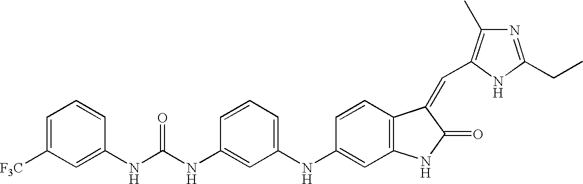 | LC-MS (m/z) [M⁺ + 1] 547.2 |
| 134 | 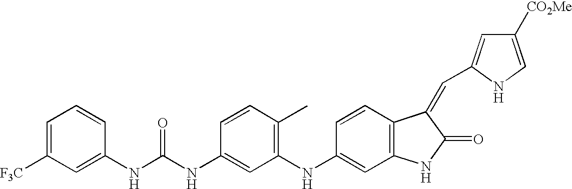 | LC-MS (m/z) [M⁺ + 1] 576.2 |
| 135 | 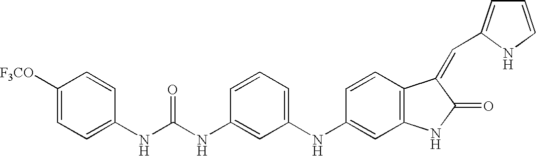 | LC-MS (m/z) [M⁺ + 1] 520.1 |
| 136 | 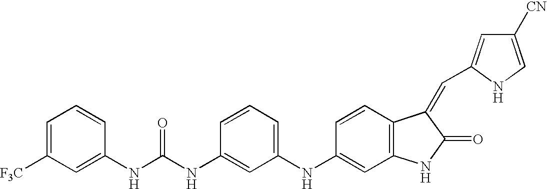 | LC-MS (m/z) [M⁺ + 1] 529.1 |
| 137 | 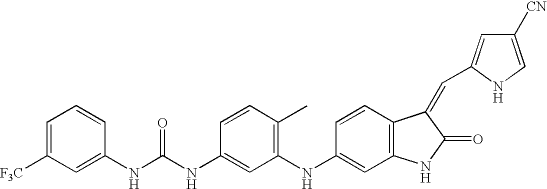 | LC-MS (m/z) [M⁺ + 1] 543.2 |
| 138 | 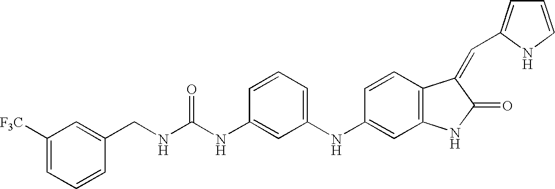 | LC-MS (m/z) [M⁺ + 1] 518.1 |
| 139 | 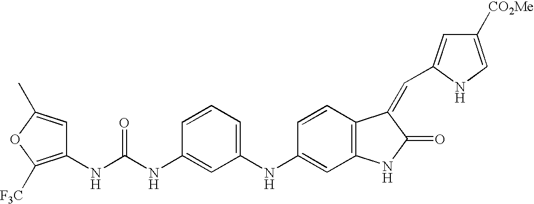 | LC-MS (m/z) [M⁺ + 1] 566.1 |
| 140 | 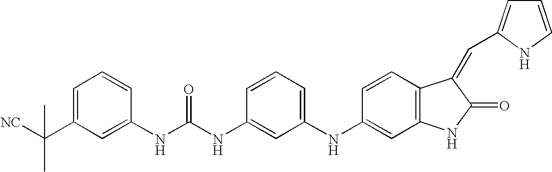 | LC-MS (m/z) [M⁺ + 1] 503.2 |
| 141 | 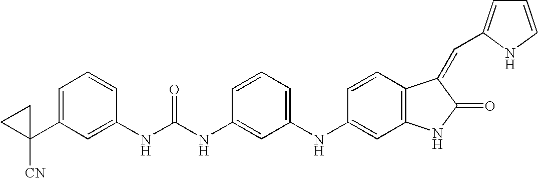 | LC-MS (m/z) [M⁺ + 1] 501.2 |
| 142 | 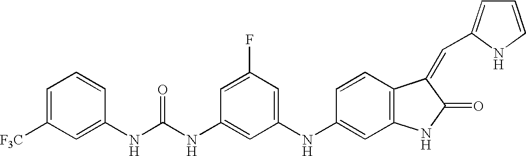 | LC-MS (m/z) [M⁺ + 1] 522.2 |
| 143 | 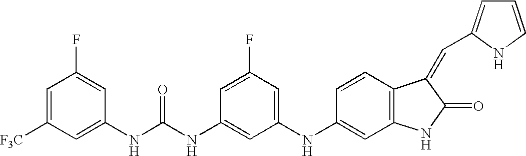 | ¹H NMR (DMSO-d₆) δ 13.14 (s, 1H), 10.69 (s, 1H), 9.29 (s, 1H), 9.04 (s, 1H), 7.95 (s, 1H), 7.72 (s, 1H), 7.62 (d, 1H), 7.52 (m, 1H), 7.55 (d, 2H), 7.25 (m, 3H), 7.13 (m, 1H), 6.69 (m, 1H), 6.51 (m, 1H), 6.30 s, 1H), 6.22 (m, 1H); LC-MS (m/z) [M⁺ + 1] 540.2 |
| 144 | 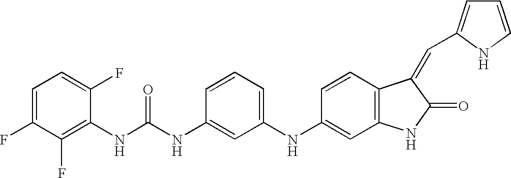 | |
| 145 | 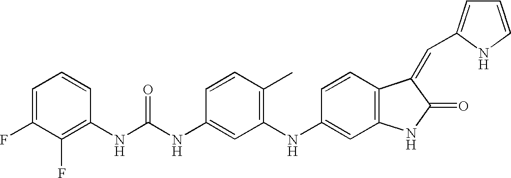 | |
| 146 | 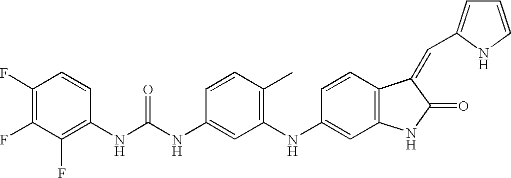 | |
| 147 | 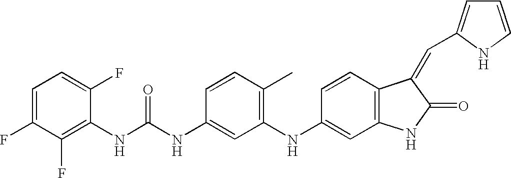 | |
| 148 | 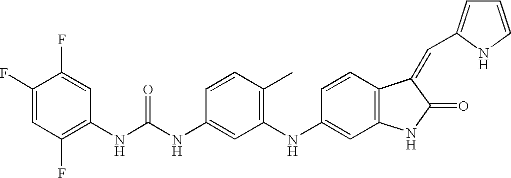 | |
| 149 | 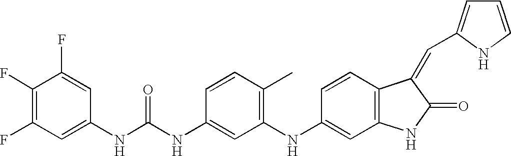 | |
| 150 | 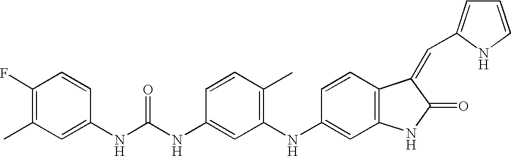 | |
| 151 | 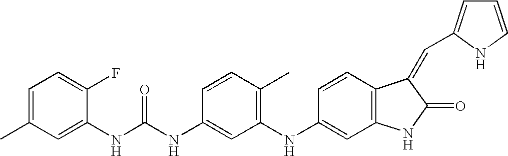 | |
| 152 | 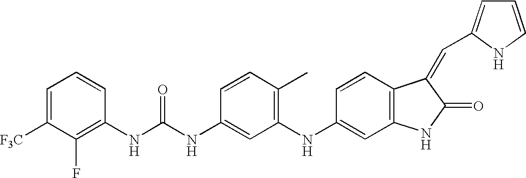 | |
| 153 | 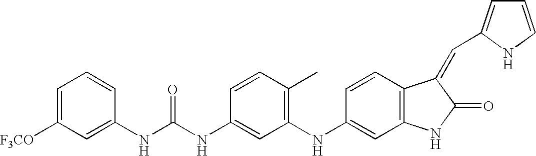 | |
| 154 | 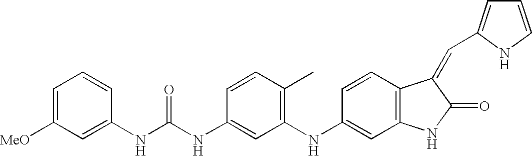 | |
| 155 | 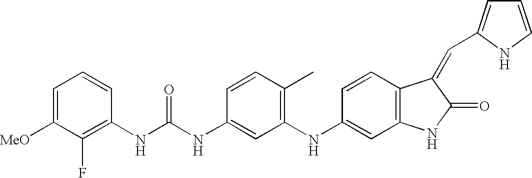 | |
| 156 | 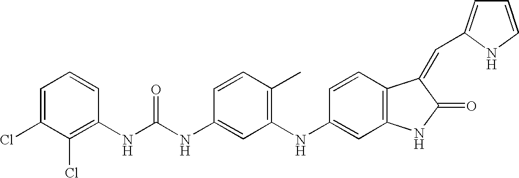 | |

### Assays

Compounds of the present invention are assayed to measure their capacity to selectively inhibit cell proliferation of Ba/F3 cells expressing Tel fusions of Trk family members, specifically ETV6-NTRK1, ETV6-NTRK2 or ETV6-NTRK3 compared with parental Ba/F3 cells.

Inhibition of Cellular TrkA, trkB or TrkC Dependent Proliferation

The cell line used is the Ba/F3 murine hematopoietic progenitor cell line transformed with human Tel-TrkA, Tel-TrkB or Tel-TrkC cDNAs (Ba/F3 EN A/B/C). These cells are maintained in RPMI/10% fetal bovine serum (RPMI/F B S) supplemented with penicillin 50 μg/mL, streptomycin 50 μg/mL and L-glutamine 200 mM. Untransformed Ba/F3 cells are similarly maintained with the addition 5 ng/ml of murine recombinant IL3.

50 μl of a Ba/F3 or Ba/F3 EN A/B/C cell suspension are plated in Greiner 384 well microplates (white) at a density of 2000 cells per well. 50 nl of serially diluted test compound (1 0-0.0001 mM in DMSO solution) is added to each well. The cells are incubated for 48 hours at 37° C., 5% CO₂. 25 μl of Bright glow is added to each well. The emited luminiscence is quantified using the Acquest™ system (Molecular Devices).

### Measuring Bioavailability of Compounds of the Invention

Five- to six-week old male Balb/c mice are housed at room temperature (18 to 22° C.) and humidity in the range 40-70%. The animal weights at the time of compound administration range from 20 to 25 grams. The mice are fed a normal diet and have free access to water at all times, before and during experiments. Fasted animals are studied on an infrequent basis. To maximize drug absorption via oral gavage and/or study food effects, animals are fasted the night before dosing and 4 hours thereafter. Animal experiments are performed according to the Animal Welfare Act and the Guide for the Care and Use of Laboratory Animals approved by the Institutional Animal Care and Use Committee (IACUC).

Test compounds are dissolved in a vehicle for dosing at a final concentration of 0.5 to 10 mg/mL. Test compounds are dosed intravenously via the lateral tail vein and orally using a gavage needle. Dosing procedures, dosing volumes, and the selection of dosing vehicles or formulations adhered to the Guidelines issued by the Novartis Pharmacology Council entitled “Preparation and Administration of Experimental Formulation in Pre-ESC Phase”. Briefly, i.v. doses are administered in solutions that are neutral and isotonic aqueous based and oral doses are administered in either solution (with or without co-solvent) or suspension.

Blood samples are drawn via retro orbital sinus. For ease of handling, animals are sometimes anesthetized under isoflurane vapor. Approximately five 50 μl samples of blood are removed each sampling time.

Pharmacokinetic parameters are calculated by non-compartmental regression analysis using Winnonlin 4.0 software (Pharsight, Mountain View, Calif., USA). The typical intravenous and oral dosing study in mice would results in the reporting of the following pharmacokinetic parameters: i.v. dosing: Vss, CL, AUC, Cₘₐₓ, Tₘₐₓ, Cₗₐₛₜ, Tₗₐₛₜ and T_{1/2}; and p.o. dosing: F, AUC, Cₘₐₓ, Tₘₐₓ, Cₗₐₛₜ, Tₗₐₛₜ and T_{1/2}.

### Effect on Proliferation of Various Kinases Dependent Cells

Compounds of the invention are tested for their antiproliferative effect on Ba/F3 cells expressing either Tel-TrkA, Tel-TrkB or Tel-TrkC and an additional panel of 34 selected diverse kinases activated by fusion to the dimerizing partners Bcr or Tel (Abl, AlK, BMX, EphA3, EphB2, FGFR2, FGFR3, FGFR4, FGR, FLT1, FLT3, FLT4, FMS, IGF1R, InsR, JAK1, JAK2, JAK3, KDR, Kit, Lck, Lyn, MER, MET, PDGFRb, RET, RON, Ros-1, Src, Syk, TIE2, TYK2, Tie1, ZAP70). The antiproliferative effect of these compounds on the different cell lines and on the non transformed cells are tested at 12 different concentrations of 3-fold serially diluted compounds in 384 well plates as described above (in media lacking IL3). The IC₅₀ values of the compounds in the different cell lines were determined from the dose response curves obtained as describe above.

### PDGFRβ

The effects of compounds of the invention on the cellular activity of PDGFRβ are conducted using Ba/F3-Tel-PDGFRβ. Compounds of the invention are tested for their ability to inhibit transformed Ba/F3-Tel-PDGFRβ cells proliferation, which is depended on PDGFRβ cellular kinase activity. Ba/F3-Tel-PDGFRβ are cultured up to 800,000 cells/mL in suspension, with RPMI 1640 supplemented with 10% fetal bovine serum as the culture medium. Cells are dispensed into 384-well format plate at 5000 cell/well in 50 μL culture medium. Compounds of the invention are dissolved and diluted in dimethylsufoxide (DMSO). Twelve points 1:3 serial dilutions are made into DMSO to create concentrations gradient ranging typically from 10 mM to 0.05 μM. Cells are added with 50 nL of diluted compounds and incubated for 48 hours in cell culture incubator. AlamarBlue® (TREK Diagnostic Systems), which can be used to monitor the reducing environment created by proliferating cells, are added to cells at final concentration of 10%. After additional four hours of incubation in a 37° C. cell culture incubator, fluorescence signals from reduced AlamarBlue® (Excitation at 530 nm, Emission at 580 nm) are quantified on Analyst GT (Molecular Devices Corp.). IC₅₀ values are calculated by linear regression analysis of the percentage inhibition of each compound at 12 concentrations.

cKit—Proliferation Assay

Compounds are tested for their ability to inhibit the proliferation of wt Ba/F3 cells and Ba/F3 cells transformed with Tel ckit fused tyrosine kinases. Untransformed Ba/F3 cells are maintained in media containing recombinant IL3. cells are plated into 384 well TC plates at 5,000 cells in 50 μl media per well and test compound at 0.06 nM to 10 μM is added. The cells are then incubated for 48 hours at 37° C., 5% CO₂. After incubating the cells, 25 μl of Bright Glo® (Promega) is added to each well following manufacturer's instructions and the plates are read using Analyst GT-Luminescence mode-50000 integration time in RLU. IC₅₀ values, the concentration of compound required for 50% inhibition, are determined from a dose response curve.

cKit—Mo7e Assay

The compounds described herein are tested for inhibition of SCF dependent proliferation using Mo7e cells which endogenously express c-kit in a 96 well format. Briefly, two-fold serially diluted test compounds (Cₘₐₓ=10 μM) are evaluated for their antiproliferative activity of Mo7e cells stimulated with human recombinant SCF. After 48 hours of incubation at 37° C., cell viability is measured by using a MTT colorimetric assay from Promega.

Compounds of Formula I, in free form or in pharmaceutically acceptable salt form exhibit valuable pharmacological properties, for example, as indicated by the in vitro tests described in this application.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 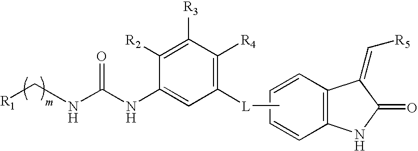
in which:
L is selected from O, NH and S;
m is selected from 0 and 1;
R₁ is selected from phenyl, pyridinyl, furanyl, isoxazolyl, pyrazolyl and thiazolyl; wherein said phenyl, pyridinyl and furanyl of R₁ can be optionally substituted with 1 to 3 radicals independently selected from halo, C₁₋₄alkyl, halo-substituted-C₁₋₄alkyl, C₁₋₄alkoxy, halo-substituted-C₁₋₄alkoxy, cyano-substituted-C₁₋₄alkyl, —XR₆ and —NR₇ₐR_{7b}; wherein X is selected from a bond and C₁₋₄alkylene; R₆ is selected from C₃₋₈heterocycloalkyl and C₃₋₁₂cycloalkyl; wherein R₆ is optionally substituted with 1 to 2 radicals independently selected from cyano and C₁₋₄alkyl; and R₇ₐ and R_{7b} are independently selected from hydrogen and C₁₋₄alkyl; wherein said isoxazolyl, pyrazolyl and thiazolyl of R₁ can be optionally substituted with 1 to 2 radicals independently selected from halo, C₁₋₄alkyl, halo-substituted-C₁₋₄alkyl, C₁₋₄alkoxy, halo-substituted-C₁₋₄alkoxy and cyano-substituted-C₁₋₄alkyl;
R₂ is selected from methyl, halo, methoxy and cyano;
R₃ is selected from methyl, halo, methoxy and cyano;
R₄ is selected from methyl, halo, methoxy and cyano;
R₅ is selected from pyrrolyl and imidazole; wherein said pyrrolyl or imidazolyl of R₅ can be optionally substituted with 1 to 2 radicals independently selected from C₁₋₄alkyl, cyano, —C(O)OR₈ₐ, —C(O)NR₈ₐR_{8b}, —X₂NR₈ₐX₂NR₈ₐR_{8b} and —C(O)NR₈ₐX₂NR₈ₐR_{8b}; wherein said alkyl substituents of R₅ are optionally substituted with —NR₉ₐR_{9b}; wherein R₈ₐ, R_{8b}, R₉ₐ and R_{9b} are each independently selected from hydrogen and C₁₋₄alkyl; each X₂ is independently C₁₋₄alkylene; and the pharmaceutically acceptable salts thereof.

2. The compound of claim 2 in which R₅ is selected from pyrrolyl and imidazolyl; wherein said pyrrolyl or imidazolyl of R₅ can be optionally substituted with 1 to 2 radicals independently selected from C₁₋₄alkyl, cyano, —C(O)OCH₃, —C(O)NH and —C(O)NH(CH₂)₂N(C₂H₅)₂; wherein said alkyl substituents of R₅ is optionally substituted with —NH₂.

3. The compound of claim 3 in which R₁ is selected from phenyl, pyridinyl, furanyl, isoxazolyl, pyrazolyl and thiazolyl; wherein said phenyl, pyridinyl and furanyl of R₁ can be optionally substituted with 1 to 3 radicals independently selected from fluoro, chloro, trifluoromethyl, methyl, ethyl, methoxy, trifluoromethoxy, difluoromethyl, 1,1-difluoroethyl, ethyl-piperazinyl-methyl, ethyl-piperazinyl, t-butyl, isopropyl, diethyl-amino-ethoxy, dimethyl-amino, 2-cyanopropan-2-yl and 1-cyanocyclopropyl; wherein said isoxazolyl, pyrazolyl and thiazolyl of R₁ can be optionally substituted with 1 to 2 radicals independently selected from fluoro, chloro, trifluoromethyl, methyl, ethyl, methoxy, trifluoromethoxy, difluoromethyl, 1,1-difluoroethyl, t-butyl, isopropyl, dimethyl-amino, 2-cyanopropan-2-yl and 1-cyanocyclopropyl.

4. The compound of claim 1 selected from: 1-{3-[2-oxo-3-(1H-pyrrol-2-ylmethylene)-2,3-dihydro-1H-indol-6-ylamino]phenyl}-3-(3-trifluoromethylphenyl)urea; 1-{3-[2-oxo-3-(1H-pyrrol-2-ylmethylene)-2,3-dihydro-1H-indol-6-ylamino]-phenyl}-3-(3,4,5-trifluorophenyl)urea; 1-{3-[2-oxo-3-(1H-pyrrol-2-ylmethylene)-2,3-dihydro-1H-indol-6-ylamino]phenyl}-3-(2,4,5-trifluorophenyl)urea; 1-{3-[2-Oxo-3-(1H-pyrrol-2-ylmethylene)-2,3-dihydro-1H-indol-6-ylamino]-phenyl}-3-phenyl-urea; 1-{4-Methyl-3-[2-oxo-3-(1H-pyrrol-2-ylmethylene)-2,3-dihydro-1H-indol-6-ylamino]-phenyl}-3-phenyl-urea; 1-{2-Methyl-5-[2-oxo-3-(1H-pyrrol-2-ylmethylene)-2,3-dihydro-1H-indol-6-ylamino]-phenyl}-3-phenyl-urea; 1-(2-Fluoro-phenyl)-3-{3-[2-oxo-3-(1H-pyrrol-2-ylmethylene)-2,3-dihydro-1H-indol-6-ylamino]-phenyl}-urea; 1-(3-Fluoro-phenyl)-3-{3-[2-oxo-3-(1H-pyrrol-2-ylmethylene)-2,3-dihydro-1H-indol-6-ylamino]-phenyl}-urea; 1-(4-Fluoro-phenyl)-3-{3-[2-oxo-3-(1H-pyrrol-2-ylmethylene)-2,3-dihydro-1H-indol-6-ylamino]-phenyl}-urea; 1-(2-Chloro-phenyl)-3-{3-[2-oxo-3-(1H-pyrrol-2-ylmethylene)-2,3-dihydro-1H-indol-6-ylamino]-phenyl}-urea; 1-(3-Chloro-phenyl)-3-{3-[2-oxo-3-(1H-pyrrol-2-ylmethylene)-2,3-dihydro-1H-indol-6-ylamino]-phenyl}-urea; 1-(4-Chloro-phenyl)-3-{3-[2-oxo-3-(1H-pyrrol-2-ylmethylene)-2,3-dihydro-1H-indol-6-ylamino]-phenyl}-urea; 1-(2,3-Difluoro-phenyl)-3-{3-[2-oxo-3-(1H-pyrrol-2-ylmethylene)-2,3-dihydro-1H-indol-6-ylamino]-phenyl}-urea; 1-(2,5-Difluoro-phenyl)-3-{3-[2-oxo-3-(1H-pyrrol-2-ylmethylene)-2,3-dihydro-1H-indol-6-ylamino]-phenyl}-urea; 1-(2,6-Difluoro-phenyl)-3-{3-[2-oxo-3-(1H-pyrrol-2-ylmethylene)-2,3-dihydro-1H-indol-6-ylamino]-phenyl}-urea; 1-(3,4-Difluoro-phenyl)-3-{3-[2-oxo-3-(1H-pyrrol-2-ylmethylene)-2,3-dihydro-1H-indol-6-ylamino]-phenyl}-urea; 1-(2,4-Difluoro-phenyl)-3-{3-[2-oxo-3-(1H-pyrrol-2-ylmethylene)-2,3-dihydro-1H-indol-6-ylamino]-phenyl}-urea; 1-{3-[2-Oxo-3-(1H-pyrrol-2-ylmethylene)-2,3-dihydro-1H-indol-6-ylamino]-phenyl}-3-(2,4,6-trifluoro-phenyl)-urea; 1-(3,5-Difluoro-phenyl)-3-{3-[2-oxo-3-(1H-pyrrol-2-ylmethylene)-2,3-dihydro-1H-indol-6-ylamino]-phenyl}-urea; 1-{3-[2-oxo-3-(1H-pyrrol-2-ylmethylene)-2,3-dihydro-1H-indol-6-ylamino]-phenyl}-3-(2,3,4-trifluoro-phenyl)-urea; 1-(2-Fluoro-phenyl)-3-{4-methyl-3-[2-oxo-3-(1H-pyrrol-2-ylmethylene)-2,3-dihydro-1H-indol-6-ylamino]-phenyl}-urea; 1-(3-Fluoro-phenyl)-3-{4-methyl-3-[2-oxo-3-(1H-pyrrol-2-ylmethylene)-2,3-dihydro-1H-indol-6-ylamino]-phenyl}-urea; 1-(2-Chloro-phenyl)-3-{4-methyl-3-[2-oxo-3-(1H-pyrrol-2-ylmethylene)-2,3-dihydro-1H-indol-6-ylamino]-phenyl}-urea; 1-(4-Fluoro-phenyl)-3-{4-methyl-3-[2-oxo-3-(1H-pyrrol-2-ylmethylene)-2,3-dihydro-1H-indol-6-ylamino]-phenyl}-urea; 1-(4-Chloro-phenyl)-3-{4-methyl-3-[2-oxo-3-(1H-pyrrol-2-ylmethylene)-2,3-dihydro-1H-indol-6-ylamino]-phenyl}-urea; 1-(3-Chloro-phenyl)-3-{4-methyl-3-[2-oxo-3-(1H-pyrrol-2-ylmethylene)-2,3-dihydro-1H-indol-6-ylamino]-phenyl}-urea; 1-{4-Methyl-3-[2-oxo-3-(1H-pyrrol-2-ylmethylene)-2,3-dihydro-1H-indol-6-ylamino]-phenyl}-3-(3-trifluoromethyl-phenyl)-urea; 1-(2,5-Difluoro-phenyl)-3-{4-methyl-3-[2-oxo-3-(1H-pyrrol-2-ylmethylene)-2,3-dihydro-1H-indol-6-ylamino]-phenyl}-urea; 1-(2,4-Difluoro-phenyl)-3-{4-methyl-3-[2-oxo-3-(1H-pyrrol-2-ylmethylene)-2,3-dihydro-1H-indol-6-ylamino]-phenyl}-urea; 1-(3,5-Difluoro-phenyl)-3-{4-methyl-3-[2-oxo-3-(1H-pyrrol-2-ylmethylene)-2,3-dihydro-1H-indol-6-ylamino]-phenyl}-urea; 1-(2,6-Difluoro-phenyl)-3-{4-methyl-3-[2-oxo-3-(1H-pyrrol-2-ylmethylene)-2,3-dihydro-1H-indol-6-ylamino]-phenyl}-urea; 1-(3,4-Difluoro-phenyl)-3-{4-methyl-3-[2-oxo-3-(1H-pyrrol-2-ylmethylene)-2,3-dihydro-1H-indol-6-ylamino]-phenyl}-urea; 1-{4-Methyl-3-[2-oxo-3-(1H-pyrrol-2-ylmethylene)-2,3-dihydro-1H-indol-6-ylamino]-phenyl}-3-(2,4,6-trifluoro-phenyl)-urea; 1-(3-Chloro-4-fluoro-phenyl)-3-{3-[2-oxo-3-(1H-pyrrol-2-ylmethylene)-2,3-dihydro-1H-indol-6-ylamino]-phenyl}-urea; 1-(4-Chloro-2-fluoro-phenyl)-3-{3-[2-oxo-3-(1H-pyrrol-2-ylmethylene)-2,3-dihydro-1H-indol-6-ylamino]-phenyl}-urea; 1-(4-Fluoro-3-methyl-phenyl)-3-{3-[2-oxo-3-(1H-pyrrol-2-ylmethylene)-2,3-dihydro-1H-indol-6-ylamino]-phenyl}-urea; 1-(2-Fluoro-5-methyl-phenyl)-3-{3-[2-oxo-3-(1H-pyrrol-2-ylmethylene)-2,3-dihydro-1H-indol-6-ylamino]-phenyl}-urea; 1-(3,5-Dimethyl-phenyl)-3-{3-[2-oxo-3-(1H-pyrrol-2-ylmethylene)-2,3-dihydro-1H-indol-6-ylamino]-phenyl}-urea; 1-(3-Ethyl-phenyl)-3-{3-[2-oxo-3-(1H-pyrrol-2-ylmethylene)-2,3-dihydro-1H-indol-6-ylamino]-phenyl}-urea; 1-(2-Fluoro-3-trifluoromethyl-phenyl)-3-{3-[2-oxo-3-(1H-pyrrol-2-ylmethylene)-2,3-dihydro-1H-indol-6-ylamino]-phenyl}-urea; 1-(3-Fluoro-5-trifluoromethyl-phenyl)-3-{3-[2-oxo-3-(1H-pyrrol-2-ylmethylene)-2,3-dihydro-1H-indol-6-ylamino]-phenyl}-urea; 1-(4-Fluoro-3-trifluoromethyl-phenyl)-3-{3-[2-oxo-3-(1H-pyrrol-2-ylmethylene)-2,3-dihydro-1H-indol-6-ylamino]-phenyl}-urea; 1-(2-Fluoro-5-trifluoromethyl-phenyl)-3-{3-[2-oxo-3-(1H-pyrrol-2-ylmethylene)-2,3-dihydro-1H-indol-6-ylamino]-phenyl}-urea; 1-{3-[2-Oxo-3-(1H-pyrrol-2-ylmethylene)-2,3-dihydro-1H-indol-6-ylamino]-phenyl}-3-(3-trifluoromethoxy-phenyl)-urea; 1-(3-Methoxy-phenyl)-3-{3-[2-oxo-3-(1H-pyrrol-2-ylmethylene)-2,3-dihydro-1H-indol-6-ylamino]-phenyl}-urea; 1-(3-Difluoromethyl-phenyl)-3-{3-[2-oxo-3-(1H-pyrrol-2-ylmethylene)-2,3-dihydro-1H-indol-6-ylamino]-phenyl}-urea; 1-(4-Fluoro-3-methoxy-phenyl)-3-{3-[2-oxo-3-(1H-pyrrol-2-ylmethylene)-2,3-dihydro-1H-indol-6-ylamino]-phenyl}-urea; 1-[3-(1,1-Difluoro-ethyl)-phenyl]-3-{3-[2-oxo-3-(1H-pyrrol-2-ylmethylene)-2,3-dihydro-1H-indol-6-ylamino]-phenyl}-urea; 1-(3-Chloro-4-fluoro-phenyl)-3-{4-methyl-3-[2-oxo-3-(1H-pyrrol-2-ylmethylene)-2,3-dihydro-1H-indol-6-ylamino]-phenyl}-urea; 1-(4-Chloro-2-fluoro-phenyl)-3-{4-methyl-3-[2-oxo-3-(1H-pyrrol-2-ylmethylene)-2,3-dihydro-1H-indol-6-ylamino]-phenyl}-urea; 1-(3,5-Dimethyl-phenyl)-3-{4-methyl-3-[2-oxo-3-(1H-pyrrol-2-ylmethylene)-2,3-dihydro-1H-indol-6-ylamino]-phenyl}-urea; 1-(3-Ethyl-phenyl)-3-{4-methyl-3-[2-oxo-3-(1H-pyrrol-2-ylmethylene)-2,3-dihydro-1H-indol-6-ylamino]-phenyl}-urea; 1-(3-Fluoro-5-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(4-Fluoro-3-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(2-Fluoro-5-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(3-Difluoromethyl-phenyl)-3-{4-methyl-3-[2-oxo-3-(1H-pyrrol-2-ylmethylene)-2,3-dihydro-1H-indol-6-ylamino]-phenyl}-urea; 1-(4-Fluoro-3-methoxy-phenyl)-3-{4-methyl-3-[2-oxo-3-(1H-pyrrol-2-ylmethylene)-2,3-dihydro-1H-indol-6-ylamino]-phenyl}-urea; 1-[3-(1,1-Difluoro-ethyl)-phenyl]-3-{4-methyl-3-[2-oxo-3-(1H-pyrrol-2-ylmethylene)-2,3-dihydro-1H-indol-6-ylamino]-phenyl}-urea; 1-(4-Methyl-3-trifluoromethyl-phenyl)-3-{3-[2-oxo-3-(1H-pyrrol-2-ylmethylene)-2,3-dihydro-1H-indol-6-ylamino]-phenyl}-urea; 1-(4-Chloro-3-trifluoromethyl-phenyl)-3-{3-[2-oxo-3-(1H-pyrrol-2-ylmethylene)-2,3-dihydro-1H-indol-6-ylamino]-phenyl}-urea; 1-(3,5-Dichloro-phenyl)-3-{3-[2-oxo-3-(1H-pyrrol-2-ylmethylene)-2,3-dihydro-1H-indol-6-ylamino]-phenyl}-urea; 1-(2,4-Dichloro-phenyl)-3-{3-[2-oxo-3-(1H-pyrrol-2-ylmethylene)-2,3-dihydro-1H-indol-6-ylamino]-phenyl}-urea; 1-(3,4-Dichloro-phenyl)-3-{3-[2-oxo-3-(1H-pyrrol-2-ylmethylene)-2,3-dihydro-1H-indol-6-ylamino]-phenyl}-urea; 1-(2,5-Dichloro-phenyl)-3-{3-[2-oxo-3-(1H-pyrrol-2-ylmethylene)-2,3-dihydro-1H-indol-6-ylamino]-phenyl}-urea; 1-(2,6-Dichloro-phenyl)-3-{3-[2-oxo-3-(1H-pyrrol-2-ylmethylene)-2,3-dihydro-1H-indol-6-ylamino]-phenyl}-urea; 1-[4-(4-Ethyl-piperazin-1-ylmethyl)-3-trifluoromethyl-phenyl]-3-{3-[2-oxo-3-(1H-pyrrol-2-ylmethylene)-2,3-dihydro-1H-indol-6-ylamino]-phenyl}-urea; 1-[3-(4-Ethyl-piperazin-1-yl)-5-trifluoromethyl-phenyl]-3-{3-[2-oxo-3-(1H-pyrrol-2-ylmethylene)-2,3-dihydro-1H-indol-6-ylamino]-phenyl}-urea; 1-(3,5-Bis-trifluoromethyl-phenyl)-3-{3-[2-oxo-3-(1H-pyrrol-2-ylmethylene)-2,3-dihydro-1H-indol-6-ylamino]-phenyl}-urea; 1-{4-Methyl-3-[2-oxo-3-(1H-pyrrol-2-ylmethylene)-2,3-dihydro-1H-indol-6-ylamino]-phenyl}-3-(4-methyl-3-trifluoromethyl-phenyl)-urea; 1-(4-Chloro-3-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(3,5-Dichloro-phenyl)-3-{4-methyl-3-[2-oxo-3-(1H-pyrrol-2-ylmethylene)-2,3-dihydro-1H-indol-6-ylamino]-phenyl}-urea; 1-(2,5-Dichloro-phenyl)-3-{4-methyl-3-[2-oxo-3-(1H-pyrrol-2-ylmethylene)-2,3-dihydro-1H-indol-6-ylamino]-phenyl}-urea; 1-(2,4-Dichloro-phenyl)-3-{4-methyl-3-[2-oxo-3-(1H-pyrrol-2-ylmethylene)-2,3-dihydro-1H-indol-6-ylamino]-phenyl}-urea; 1-(3,4-Dichloro-phenyl)-3-{4-methyl-3-[2-oxo-3-(1H-pyrrol-2-ylmethylene)-2,3-dihydro-1H-indol-6-ylamino]-phenyl}-urea; 1-(3,5-Bis-trifluoromethyl-phenyl)-3-{4-methyl-3-[2-oxo-3-(1H-pyrrol-2-ylmethylene)-2,3-dihydro-1H-indol-6-ylamino]-phenyl}-urea; 1-(2,5-Dimethyl-furan-3-yl)-3-{3-[2-oxo-3-(1H-pyrrol-2-ylmethylene)-2,3-dihydro-1H-indol-6-ylamino]-phenyl}-urea; 1-(5-tert-Butyl-2-methyl-furan-3-yl)-3-{3-[2-oxo-3-(1H-pyrrol-2-ylmethylene)-2,3-dihydro-1H-indol-6-ylamino]-phenyl}-urea; 1-{2-Methyl-5-[2-oxo-3-(1H-pyrrol-2-ylmethylene)-2,3-dihydro-1H-indol-6-ylamino]-phenyl}-3-(3-trifluoromethyl-phenyl)-urea; 1-(2-Fluoro-5-trifluoromethyl-phenyl)-3-{2-methyl-5-[2-oxo-3-(1H-pyrrol-2-ylmethylene)-2,3-dihydro-1H-indol-6-ylamino]phenyl}-urea; 1-{4-Methoxy-3-[2-oxo-3-(1H-pyrrol-2-ylmethylene)-2,3-dihydro-1H-indol-6-ylamino]-phenyl}-3-(3-trifluoromethyl-phenyl)-urea; 1-(2-Fluoro-5-trifluoromethyl-phenyl)-3-{4-methoxy-3-[2-oxo-3-(1H-pyrrol-2-ylmethylene)-2,3-dihydro-1H-indol-6-ylamino]-phenyl}-urea; 1-{4-Fluoro-3-[2-oxo-3-(1H-pyrrol-2-ylmethylene)-2,3-dihydro-1H-indol-6-ylamino]-phenyl}-3-(2-fluoro-5-trifluoromethyl-phenyl)-urea; 1-{4-Fluoro-3-[2-oxo-3-(1H-pyrrol-2-ylmethylene)-2,3-dihydro-1H-indol-6-ylamino]-phenyl}-3-(3-trifluoromethyl-phenyl)-urea; 1-{2-Fluoro-5-[2-oxo-3-(1H-pyrrol-2-ylmethylene)-2,3-dihydro-1H-indol-6-ylamino]-phenyl}-3-(3-trifluoromethyl-phenyl)-urea; 1-{2-Fluoro-5-[2-oxo-3-(1H-pyrrol-2-ylmethylene)-2,3-dihydro-1H-indol-6-ylamino]-phenyl}-3-(2-fluoro-5-trifluoromethyl-phenyl)-urea; 1-(2-Chloro-5-trifluoromethyl-phenyl)-3-{2-methyl-5-[2-oxo-3-(1H-pyrrol-2-ylmethylene)-2,3-dihydro-1H-indol-6-ylamino]-phenyl}-urea; 1-(2-Chloro-5-trifluoromethyl-phenyl)-3-{4-methoxy-3-[2-oxo-3-(1H-pyrrol-2-ylmethylene)-2,3-dihydro-1H-indol-6-ylamino]-phenyl}-urea; 1-(2-Chloro-5-trifluoromethyl-phenyl)-3-{2-fluoro-5-[2-oxo-3-(1H-pyrrol-2-ylmethylene)-2,3-dihydro-1H-indol-6-ylamino]-phenyl}-urea; 1-{4-Methyl-3-[2-oxo-3-(1H-pyrrol-2-ylmethylene)-2,3-dihydro-1H-indol-6-ylamino]-phenyl}-3-m-tolyl-urea; 1-{3-[2-Oxo-3-(1H-pyrrol-2-ylmethylene)-2,3-dihydro-1H-indol-6-ylamino]-phenyl}-3-m-tolyl-urea; 1-(3-Isopropyl-phenyl)-3-{3-[2-oxo-3-(1H-pyrrol-2-ylmethylene)-2,3-dihydro-1H-indol-6-ylamino]-phenyl}-urea; 1-(3-tert-Butyl-phenyl)-3-{3-[2-oxo-3-(1H-pyrrol-2-ylmethylene)-2,3-dihydro-1H-indol-6-ylamino]-phenyl}-urea; 1-{3-Cyano-5-[2-oxo-3-(1H-pyrrol-2-ylmethylene)-2,3-dihydro-1H-indol-6-ylamino]-phenyl}-3-(2-fluoro-5-trifluoromethyl-phenyl)-urea; 1-(3-Fluoro-5-trifluoromethyl-phenyl)-3-{3-[2-oxo-3-(1H-pyrrol-2-ylmethylene)-2,3-dihydro-1H-indol-6-ylsulfanyl]-phenyl}-urea; 1-(2-Fluoro-5-trifluoromethyl-phenyl)-3-{3-[2-oxo-3-(1H-pyrrol-2-ylmethylene)-2,3-dihydro-1H-indol-6-ylsulfanyl]-phenyl}-urea; 1-{3-Cyano-5-[2-oxo-3-(1H-pyrrol-2-ylmethylene)-2,3-dihydro-1H-indol-6-ylamino]-phenyl}-3-(3-trifluoromethyl-phenyl)-urea; 1-{3-[2-Oxo-3-(1H-pyrrol-2-ylmethylene)-2,3-dihydro-1H-indol-6-ylsulfanyl]-phenyl}-3-(3-trifluoromethyl-phenyl)-urea; 1-{3-Cyano-5-[2-oxo-3-(1H-pyrrol-2-ylmethylene)-2,3-dihydro-1H-indol-6-ylamino]-phenyl}-3-(3-fluoro-5-trifluoromethyl-phenyl)-urea; 1-(2-Chloro-5-trifluoromethyl-phenyl)-3-{4-fluoro-3-[2-oxo-3-(1H-pyrrol-2-ylmethylene)-2,3-dihydro-1H-indol-6-ylamino]-phenyl}-urea; 1-{4-Fluoro-3-[2-oxo-3-(1H-pyrrol-2-ylmethylene)-2,3-dihydro-1H-indol-6-ylamino]-phenyl}-3-(3-trifluoromethoxy-phenyl)-urea; 1-{2-Fluoro-5-[2-oxo-3-(1H-pyrrol-2-ylmethylene)-2,3-dihydro-1H-indol-6-ylamino]-phenyl}-3-(3-trifluoromethoxy-phenyl)-urea; 1-(3-Chloro-phenyl)-3-{3-[3-(5-methyl-3H-imidazol-4-ylmethylene)-2-oxo-2,3-dihydro-1H-indol-6-ylamino]-phenyl}-urea; 1-{3-[2-oxo-3-(1H-pyrrol-2-ylmethylene)-2,3-dihydro-1H-indol-5-ylamino]-phenyl}-3-phenyl-urea; 1-{3-[2-oxo-3-(1H-pyrrol-2-ylmethylene)-2,3-dihydro-1H-indol-6-ylamino]-phenyl}-3-(2-trifluoromethyl-phenyl)-urea; 1-{3-[2-oxo-3-(1H-pyrrol-2-ylmethylene)-2,3-dihydro-1H-indol-6-ylamino]-phenyl}-3-(4-trifluoromethyl-phenyl)-urea; 1-{3-[3-(4-Cyano-1H-pyrrol-2-ylmethylene)-2-oxo-2,3-dihydro-1H-indol-6-ylamino]-phenyl}-3-phenyl-urea; 1-(4-Fluoro-phenyl)-3-{3-[3-(5-methyl-3H-imidazol-4-ylmethylene)-2-oxo-2,3-dihydro-1H-indol-6-ylamino]-phenyl}-urea; 5-(6-{3-[3-(3-Fluoro-phenyl)-ureido]-phenylamino}-2-oxo-1,2-dihydro-indol-3-ylidenemethyl)-1H-pyrrole-3-carboxylic acid; 5-(6-{3-[3-(3-Fluoro-phenyl)-ureido}-phenylamino]-2-oxo-1,2-dihydro-indol-3-ylidenemethyl)-1H-pyrrole-3-carboxylic acid (2-diethylamino-ethyl)-amide; 1-(4-Dimethylamino-phenyl)-3-{3-[2-oxo-3-(1H-pyrrol-2-ylmethylene)-2,3-dihydro-1H-indol-6-ylamino]-phenyl}-urea; 1-(3-Fluoro-phenyl)-3-{3-[3-(4-methyl-1H-imidazol-2-ylmethylene)-2-oxo-2,3-dihydro-1H-indol-6-ylamino]-phenyl}-urea; 5-(6-{3-[3-(4-Fluoro-phenyl)-ureido]-phenylamino}-2-oxo-1,2-dihydro-indol-3-ylidenemethyl)-1H-pyrrole-3-carboxylic acid; 1-{3-Methoxy-5-[2-oxo-3-(1H-pyrrol-2-ylmethylene)-2,3-dihydro-1H-indol-6-ylamino]-phenyl}-3-phenyl-urea; 1-(3-Chloro-phenyl)-3-{3-[3-(4-methyl-1H-imidazol-2-ylmethylene)-2-oxo-2,3-dihydro-1H-indol-6-ylamino]-phenyl}-urea; 1-(3-Chloro-phenyl)-3-{3-[3-(2ethyl-5-methyl-3H-imidazol-4-ylmethylene)-2-oxo-2,3-dihydro-1H-indol-6-ylamino]-phenyl}-urea; 1-(2,6-Dichloro-pyridin-4-yl)-3-{3-[2-oxo-3-(1H-pyrrol-2-ylmethylene)-2,3-dihydro-1H-indol-6-ylamino]-phenyl}-urea; 1-(5-Methyl-2-trifluoromethyl-furan-3-yl)-3-{3-[2-oxo-3-(1H-pyrrol-2-ylmethylene)-2,3-dihydro-1H-indol-6-ylamino]-phenyl}-urea; 1-{3-[2-oxo-3-(1H-pyrrol-2-ylmethylene)-2,3-dihydro-1H-indol-6-ylamino]-phenyl}-3-pyridin-3-yl-urea; 5-(2-oxo-6-{3-[3-(3-trifluoromethyl-phenyl)-ureido]-phenylamino}-1,2-dihydro-indol-3-ylidenemethyl)-1H-pyrrole-3-carboxylic acid; 5-(2-oxo-6-{3-[3-(3-trifluoromethyl-phenyl)-ureido]-phenylamino}-1,2-dihydro-indol-3-ylidenemethyl)-1H-pyrrole-3-carboxylic acid methyl ester; 1-{3-[3-(4-Methyl-1H-imidazol-2-ylmethylene)-2-oxo-2,3-dihydro-1H-indol-6-ylamino]-phenyl}-3-(3-trifluoromethyl-phenyl)-urea; 1-{3-[3-(2-Ethyl-5-methyl-3H-imidazol-4-ylmethylene)-2-oxo-2,3-dihydro-1H-indol-6-ylamino]-phenyl}-3-(3-trifluoromethyl-phenyl)-urea; 5-(6-{2-Methyl-5-[3-(3-trifluoromethyl-phenyl)-ureido]-phenylamino}-2-oxo-1,2-dihydro-indol-3-ylidenemethyl)-1H-pyrrole-3-carboxylic acid methyl ester; 1-{3-[2-oxo-3-(1H-pyrrol-2-ylmethylene)-2,3-dihydro-1H-indol-6-ylamino]-phenyl}-3-(4-trifluoromethoxy-phenyl)-urea; 1-{3-[3-(4-Cyano-1H-pyrrol-2-ylmethylene)-2-oxo-2,3-dihydro-1H-indol-6-ylamino]-phenyl}-3-(3-trifluoromethyl-phenyl)-urea; 1-{3-[3-(4-Cyano-1H-pyrrol-2-ylmethylene)-2-oxo-2,3-dihydro-1H-indol-6-ylamino]-4-methyl-phenyl}-3-(3-trifluoromethyl-phenyl)-urea; 1-{3-[2-oxo-3-(1H-pyrrol-2-ylmethylene)-2,3-dihydro-1H-indol-6-ylamino]-phenyl}-3-(3-trifluoromethyl-benzyl)-urea; 5-(6-{3-[3-(5-Methyl-2-trifluoromethyl-furan-3-yl)-ureido]-phenylamino}-2-oxo-1,2-dihydro-indol-3-ylidenemethyl)-1H-pyrrole-3-carboxylic acid methyl ester; 1-[3-(Cyano-dimethyl-methyl)-phenyl]-3-{3-[2-oxo-3-(1H-pyrrol-2-ylmethylene)-2,3-dihydro-1H-indol-6-ylamino]-phenyl}-urea; 1-[3-(1-Cyano-cyclopropyl)-phenyl]-3-{3-[2-oxo-3-(1H-pyrrol-2-ylmethylene)-2,3-dihydro-1H-indol-6-ylamino]-phenyl}-urea; 1-{3-Fluoro-5-[2-oxo-3-(1H-pyrrol-2-ylmethylene)-2,3-dihydro-1H-indol-6-ylamino]-phenyl}-3-(3-trifluoromethyl-phenyl)-urea; and 1-(2,5-Dichloro-phenyl)-3-{4-fluoro-3-[2-oxo-3-(1H-pyrrol-2-ylmethylene)-2,3-dihydro-1H-indol-6-ylamino]-phenyl}-urea.

5. A pharmaceutical composition comprising a therapeutically effective amount of a compound of claim 1 in combination with a pharmaceutically acceptable excipient.

6. A method for treating a disease in an animal in which inhibition of kinase activity can prevent, inhibit or ameliorate the pathology and/or symptamology of the disease, which method comprises administering to the animal a therapeutically effective amount of a compound of claim 1.

7. The method of claim 6 in which the kinase is selected from the group consisting of TrkA, TrkB and TrkC.

8. The method of claim 7 in which the disease is pancreatic cancer.

9. The method of claim 7 in which the disease is papillary thyroid carcinoma.

10. The method of claim 7 in which the disease is neuroblastoma.

11. The method of claim 6 in which the kinase is selected from PDGFR and c-kit.
